(19) United States
(12) Patent Application Publication
(10) Pub. No.: US 2020/0251226 A1
(43) Pub. Date: Aug. 6, 2020

(54) METHOD AND SYSTEM FOR SCHEDULING AND DOCUMENT-SHARING WITHIN AN ENTERPRISE VIRTUAL HEALTH NETWORK

(71) Applicants: Alex Vo, Denver, CO (US); Mari Robinson, Galveston, TX (US); Oliver Black, Galveston, TX (US); Ryan S. Westberry, Denver, CO (US); David L. Callender, Galveston, TX (US)

(72) Inventors: Alex Vo, Denver, CO (US); Mari Robinson, Galveston, TX (US); Oliver Black, Galveston, TX (US); Ryan S. Westberry, Denver, CO (US); David L. Callender, Galveston, TX (US)

(73) Assignee: Board of Regents, The University of Texas System, Austin, TX (US)

(21) Appl. No.: 16/780,737

(22) Filed: Feb. 3, 2020

Related U.S. Application Data
(60) Provisional application No. 62/801,000, filed on Feb. 4, 2019.

(51) Int. Cl. G16H 80/00; G06Q 10/10; G16H 10/60

(52) U.S. Cl. CPC G16H 80/00; G16H 10/60; G06Q 10/1093

(57) ABSTRACT

A system and method for scheduling and tracking a telemedicine encounter includes a database of resource pool profiles comprising healthcare provider profiles, telemedicine location profiles, and patient profile(s), receiving a telemedicine scheduling request comprising a patient data and appointment parameter(s) from a referring healthcare provider device (HPD), searching the resource pool profiles for medical provider profiles matching the appointment parameter(s), providing matching appointment time(s) to the referring HPD, receiving a selected appointment time from the referring HPD, automatically sending a booking alert to the referring and referred to HPDs, scheduling the telemedicine encounter for the healthcare provider profile and telemedicine location profile at the selected appointment time, creating an appointment timeline comprising appointment task(s), automatically creating and providing videoconference link(s) to the referring and referred to HPDs, and automatically tracking and sending alerts upon completion of each telemedicine encounter task.

### CROSS-REFERENCE TO RELATED APPLICATIONS

This patent application claims priority to U.S. Provisional Patent Application No. 62/801,000 filed on Feb. 4, 2019 and entitled “Method and System for Scheduling and Document-Sharing within an Enterprise Virtual Health Network”, the contents of which are hereby incorporated by reference in their entirety.

### TECHNICAL FIELD OF THE INVENTION

The present invention relates generally to the field of information systems, more specifically, information systems relating to scheduling and document storage, distribution, and control.

### INCORPORATION-BY-REFERENCE OF MATERIALS FILED ON COMPACT DISC

None.

### STATEMENT OF FEDERALLY FUNDED RESEARCH

None.

### BACKGROUND OF INVENTION

Telemedicine has been part of the healthcare landscape for the past 50-60 years and has been integrated in all kinds of applications for patient care. It is now becoming a standard part of normal operations in both behavioral and physical healthcare systems. Telemedicine offers a variety of important benefits to the healthcare system such as improved service access, cost efficacy, and improved service quality.

In addition to serving clients in distant areas, telemedicine has been associated with improved access to care for special populations including disabled, elderly, correctional, and other mobility-challenged persons. Further, it has improved access to specialized care by expanding the reach of specialists to persons beyond their own geographical locations.

John Hopkins University developed a “Hospital at Home” model using telemedicine for Medicaid and Medicare Advantage members and found a 19% savings over similar patients using traditional in-patient services. Savings were attributed to shorter stays for in-patient services and fewer labs and diagnostics. Similar findings were reported for a chronically ill Medicare sample. As treatment for the chronically ill accounts for almost 80% of U.S. healthcare spending, this is a particularly important opportunity for telemedicine.

A variety of other studies cite cost savings ranging from 8% to 25% as compared to matched in-patient comparisons, and many attribute the savings to earlier identification of acute issues and shorter hospital stays when in-patient care was required. Limiting in-patient stays may also reduce the incidence of further complications for vulnerable populations for whom the hospital environment actually increases health risk via exposure to communicable disease. Other investigations link telemedicine to emergency department diversions, which is associated with cost savings and mitigation of transportation risks for vulnerable populations such as the elderly.

While some studies show that telemedicine is as effective as traditional face-to-face clinical service, other studies have found higher rates of clinical success with telemedicine service delivery. Examples include reduced hospital admissions and re-admissions, reductions in symptoms for chronically ill patients (specifically symptoms of heart disease, diabetes, Parkinson's disease, and psychiatric distress), improved emergency room mortality rates, and improved Global Assessment of Functioning psychological ratings.

Studies analyzing the use of telemedicine for assessments and screenings have also found equal or improved quality results as compared to matched, in-person assessments. Some hypothesize that assessments may be more accurate when the patient is able to remain in a familiar environment. Examples include observational assessments of emotionally-behaviorally disturbed youth in their typical home/school environment and improved disclosure by adult patients who may provide more honest disclosure while in a more comfortable environment.

Patient perspective is also critical to any assessment of service quality. Studies including measures of patient satisfaction cited satisfaction ratings from 85% to 96%, which were equal to, or in some cases greater than satisfaction ratings for matched in-patient comparisons. Also, telehealth options present a level of convenience for patients in the sense that minor health concerns can be addressed faster and typically while still at home. In some instances, virtual care programs can provide patients with instant access to a doctor or physician—no wait involved. Other programs will be able to connect patients with physicians during off or non-regular office hours, giving patients immediate access without having to go to the emergency room.

The state of Texas has many confounding characteristics that make telemedicine a great option to its already taxed healthcare system. With a land mass area of 268,820 square miles and a growing population of 25.1 million, Texas is the second largest US state both by area and population. Its population growth rose more than 20% in the past decade. This rapid growth is attributed to a diversity of sources such as natural increases from the total of all births minus all deaths, and to a high rate of net in-migration from other states and countries. Along with the increase in population, an ever-growing aging population has significantly affected the demand on the healthcare workforce as demands for quality care increased.

In its Statewide Health Plan, the Texas Statewide Health Council concluded: Texas faces particular challenges with respect to physician and other healthcare workforces not primarily because of an overall shortage, but because of sharp disparities in the allocation of healthcare resources to different parts of the state. In the metropolitan areas outside the border, there is one physician in direct patient care for each 573 county residents. In the 32 county border region and in non-metropolitan Texas, the ratios are 2 to 3 times as high.

Although the overall supply of physicians has increased in Texas since 2000 from in-migration, the vast majority of these healthcare professionals resides and practices within 4 primary areas of Texas: Dallas, Houston, Austin, and San Antonio. Moreover, Texas has consistently lagged behind the US average in the ratio of physician supply per 100,000 of population, and the gap between the two appears to be increasing.

Theoretically, resources such as healthcare would be distributed across the state in accordance with population density and needs. Realistically, however, geographical and economic barriers create significant disparities across the state, with rural and underserved communities enduring significantly greater barriers to accessing the care continuum. The supply ratios for a number of health professionals, including primary care physicians and mental health professionals, are lowest in rural, border and other health professional shortage areas.

In most communities, especially in rural areas, care is not organized to promote prevention and early intervention, coordinate services, or monitor access to and quality of care. Moreover, public and private funding to subsidize care remains inadequate, despite growing community needs associated with increases in the uninsured and aging populations. Consequently, many people are left to seek care in emergency rooms, often as a last resort, in an unmanaged and episodic manner. The costs of such care are borne by care-giving institutions, local governments, and, ultimately, taxpayers, many of whom are already burdened with the costs of meeting health-related costs of their own.

In response to the shortage of medical specialty providers across the state of Texas, the University of Texas System (UT) initiated a bold project to leverage the size of its academic medical centers to create the UT Virtual Health Network (VHN). The VEIN is designed to create a telehealth infrastructure connecting all eight of the UT Health Science Centers and Medical Schools as well as to other academic medical centers as a mechanism to span crucial specialty services across the state.

The VHN provides coordinated outbound and inbound (from connected hubs) support for telemedical services from all eight of the University of Texas Health Science Centers and Medical Schools into care settings such as other hospitals and clinics, nursing facilities, schools, employee work sites, and patient residences. In future program expansion, the VEIN will also be able to partner and provide medical services from hospitals and organizations outside of the University of Texas System, which may include other Texas based health science institutions such as Baylor Healthcare System, Texas Tech Health Sciences Center, University of North Texas, Children's Health System, and Texas A&M University.

As part of the VHN construct, each UT Health Science Center and Medical School will be designated as a Hub, leveraging its areas of clinical expertise to serve patients that normally would not have access to the individual healthcare facilities, and capitalizing on world-class expertise distributed across the state. An important feature of the VEIN construct is a centralized approach to administration while empowering regional and local healthcare facilities to expand their clinical service offerings by utilizing telehealth technologies to access a multiplicity of clinical care services across institutions.

### SUMMARY OF THE INVENTION

The scheduler and document-sharing platform features built in algorithms that allow users to schedule telemedicine and in-office appointments as well as exchange patient information. Based on information entered, the system can pinpoint corresponding physicians, appropriate facilities and available times for scheduling patient consultations. The scheduler also tracks user input, appointment start/stop times and other meta data to allow gathering of statistics for analysis of system efficiency and pinpointing possible issues. Patient files may be exchanged by utilizing the document-sharing embodiments of the platform.

One embodiment of the current invention provides a computerized method of scheduling and tracking a telemedicine encounter between a referring healthcare provider and a referred to healthcare provider, comprising: providing an input/output interface, a memory one or more processors communicably coupled to the input/output interface and the memory; providing a database of resource pool profiles stored in the at least one memory unit comprising two or more healthcare provider profiles, two or more telemedicine location profiles, and one or more patient profiles, wherein each healthcare provider profile is linked to one or more of the telemedicine location profiles and one or more medical specialties; receiving a telemedicine scheduling request for a patient from a referring healthcare provider device, the scheduling request comprising a patient data and one or more appointment parameters; searching the database of resource pool profiles for any medical provider profiles that match the one or more appointment parameters; providing one or more appointment times that match the one or more appointment parameters to the referring healthcare provider device, wherein each appointment time is linked to one or more of the healthcare provider profiles; receiving a selected appointment time from the one or more appointment times from the referring healthcare provider device; automatically sending a booking alert to the referring healthcare provider device and a referred to healthcare provider device associated with the selected appointment time, scheduling the telemedicine encounter for the healthcare provider profile and the telemedicine location profile at the selected appointment time, and creating an appointment timeline for the patient comprising a set of appointment tasks; automatically creating and providing one or more videoconference links for the telemedicine encounter to the referring healthcare provider device and the referred to healthcare provider device; and automatically tracking and sending alerts upon completion of each of the set of telemedicine encounter tasks.

In one aspect, the one or more appointment parameters comprise a patient location, a requested telemedicine location selected from the telemedicine location profiles, a requested medical specialty selected from the one or more medical specialties, a requested medical profile selected from the two or more medical profiles, a requested appointment length, a requested medical provider gender, an appointment urgency, a request for one or more medical peripherals, or a combination thereof. In another aspect, the method further comprises assigning a role designation to a user, wherein the role designation comprises an institutional administrator, a department administrator, a technical administrator, a presenter, a healthcare provider, or a referring healthcare provider. In another aspect, the method further comprises providing access to the computer system based on a hierarchical structure of the role assignment, an access and a function of the user. In another aspect, the referring healthcare provider device and the referred to healthcare provider device comprise one or more of a computer, a laptop, a handheld device, or a mobile device; and the referring healthcare provider and the referred to healthcare provider each comprise one or more of a physician, nurse practitioner, physician assistance, nurse, nurse's aid, other healthcare professional, a healthcare coordinator or a healthcare staff. In another aspect, the referring healthcare provider device comprises more than one device; or the referred to healthcare provider device comprises more than one device. In another aspect, the set of telemedicine encounter tasks comprise specifying a payment method, sending a request for a pre-appointment patient medical information, receiving the pre-appointment patient medical information, confirming the payment method, initiating the telemedicine encounter, completing the telemedicine encounter, sending the patient results, or receiving the patient results. In another aspect, the method further comprises one or more of the following: receiving a payment method from the referring healthcare provider device; receiving a confirmation of the payment method from the referred to healthcare provider; sending a request for a pre-appointment patient medical information to the referring healthcare provider device; receiving the pre-appointment patient medical information from the referring healthcare provider device; initiating the telemedicine encounter using the one or more videoconference links; receiving a completion of the telemedicine encounter from the referred to healthcare provider device; receiving the patient results from the referred to healthcare provider device; or providing the patient results to the referring healthcare provider device. In another aspect, the method further comprises automatically ranking the one or more appointment times based on one or more criteria comprising a capacity at the two or more telemedicine location profiles, the two or more healthcare provider profiles, or a utilization across a set of the resource pool profiles. In another aspect, the method further comprises sharing one or more documents or files between the referring healthcare provider device and the referred to healthcare provider device via the computer system. In another aspect, the method further comprises using the one or more documents or files for clinical support, administrative support, education, tutoring, training, credentialing of one or more of the resource pool profiles, or store and forward telemedicine consultations. In another aspect, the method further comprises storing, distributing and processing the one or more documents for files in the telemedicine encounter or an evaluation of the telemedicine encounter. In another aspect, the method further comprises receiving one or more documents or files from the referring healthcare provider device or the referred to healthcare provider device; making the received one or more documents or files available for downloading to the referring healthcare provider device or the referred to healthcare provider device; and removing the received one or more documents or files from the computer system after a preset amount of time or until the received one or more documents or files are downloaded. In another aspect, the resource pool profiles comprise healthcare facility profiles, healthcare department profiles, healthcare unit profiles, or healthcare organization profiles. In another aspect, the telemedicine location profiles comprise patient stations with video conference capabilities, and patient station peripherals for specific patient healthcare evaluations. In another aspect, the method further comprises providing an assignment of the resource pool profiles in a list or calendar format. In another aspect, the computerized system is integrated into or communicably linked to an electronic medical record (EMR) system.

Another embodiment of the current invention provides a system for scheduling and tracking a telemedicine encounter between a referring healthcare provider and a referred to healthcare provider, comprising: an input/output interface; a memory; one or more processors communicably coupled to the input/output interface and the memory; a database of resource pool profiles stored in the memory comprising two or more healthcare provider profiles, two or more telemedicine location profiles, and one or more patient profiles, wherein each healthcare provider profile is linked to one or more of the telemedicine location profiles and one or more medical specialties; and the one or more processors: receive a telemedicine scheduling request for a patient from a referring healthcare provider device via the input/output interface, the scheduling request comprising a patient data and one or more appointment parameters, search the database of resource pool profiles for any medical provider profiles that match the one or more appointment parameters, provide one or more appointment times that match the one or more appointment parameters to the referring healthcare provider device via the input/output interface, wherein each appointment time is linked to one or more of the healthcare provider profiles, receive a selected appointment time from the one or more appointment times from the referring healthcare provider device via the input/output interface, automatically send a booking alert to the referring healthcare provider device and a referred to healthcare provider device associated with the selected appointment time via the input/output interface, schedule the telemedicine encounter for the healthcare provider profile and the telemedicine location profile at the selected appointment time, and create an appointment timeline for the patient comprising a set of appointment tasks, automatically create and provide one or more videoconference links for the telemedicine encounter to the referring healthcare provider device and the referred to healthcare provider device via the input/output interface, and automatically track and send alerts upon completion of each of the set of telemedicine encounter tasks.

In one aspect, the one or more appointment parameters comprise a patient location, a requested telemedicine location selected from the telemedicine location profiles, a requested medical specialty selected from the one or more medical specialties, a requested medical profile selected from the two or more medical profiles, a requested appointment length, a requested medical provider gender, an appointment urgency, a request for one or more medical peripherals, or a combination thereof. In another aspect, the one or more processors assign a role designation to a user, wherein the role designation comprises an institutional administrator, a department administrator, a technical administrator, a presenter, a healthcare provider, or a referring healthcare provider. In another aspect, the one or more processors provide access to the computer system based on a hierarchical structure of the role assignment, an access and a function of the user. In another aspect, the referring healthcare provider device and the referred to healthcare provider device comprise one or more of a computer, a laptop, a handheld device, or a mobile device; and the referring healthcare provider and the referred to healthcare provider each comprise one or more of a physician, nurse practitioner, physician assistance, nurse, nurse's aid, other healthcare professional, a healthcare coordinator or a healthcare staff. In another aspect, the referring healthcare provider device comprises more than one device; or the referred to healthcare provider device comprises more than one device. In another aspect, the set of telemedicine encounter tasks comprise specifying a payment method, sending a request for a pre-appointment patient medical information, receiving the pre-appointment patient medical information, confirming the payment method, initiating the telemedicine encounter, completing the telemedicine encounter, sending the patient results, or receiving the patient results. In another aspect, the one or more processors perform one or more of the following: receive a payment method from the referring healthcare provider device; receive a confirmation of the payment method from the referred to healthcare provider; send a request for a pre-appointment patient medical information to the referring healthcare provider device; receive the pre-appointment patient medical information from the referring healthcare provider device; initiate the telemedicine encounter using the one or more videoconference links; receive a completion of the telemedicine encounter from the referred to healthcare provider device; receive the patient results from the referred to healthcare provider device; or provide the patient results to the referring healthcare provider device. In another aspect, the one or more processors automatically rank the one or more appointment times based on one or more criteria comprising a capacity at the two or more telemedicine location profiles, the two or more healthcare provider profiles, or a utilization across a set of the resource pool profiles. In another aspect, the one or more processors share one or more documents or files between the referring healthcare provider device and the referred to healthcare provider device via the computer system. In another aspect, the one or more processors use the one or more documents or files for clinical support, administrative support, education, tutoring, training, credentialing of one or more of the resource pool profiles, or store and forward telemedicine consultations. In another aspect, the one or more processors store, distribute and process the one or more documents for files in the telemedicine encounter or an evaluation of the telemedicine encounter. In another aspect, the one or more processors: receive one or more documents or files from the referring healthcare provider device or the referred to healthcare provider device; make the received one or more documents or files available for downloading to the referring healthcare provider device or the referred to healthcare provider device; and remove the received one or more documents or files from the computer system after a preset amount of time or until the received one or more documents or files are downloaded. In another aspect, the resource pool profiles comprise healthcare facility profiles, healthcare department profiles, healthcare unit profiles, or healthcare organization profiles. In another aspect, the telemedicine location profiles comprise patient stations with video conference capabilities, and patient station peripherals for specific patient healthcare evaluations. In another aspect, the one or more processors provide an assignment of the resource pool profiles in a list or calendar format. In another aspect, the system is integrated into or communicably linked to an electronic medical record (EMR) system.

The present invention is described in detail below with reference to the accompanying drawings.

### BRIEF DESCRIPTION OF THE DRAWINGS

The above and further advantages of the invention may be better understood by referring to the following descriptions in conjunction with the accompanying drawings, in which:

FIG. 1 is a block diagram of a system according to an embodiment of the current invention;

FIG. 2 is a flow chart of a method in according to an embodiment of the current invention;

FIG. 3 is an illustrative schematic of a virtual health network scheduler and document-sharing platform according to an embodiment of the current invention;

FIG. 4 is an example of the institution selection drop down menu according to an embodiment of the current invention;

FIG. 5 is an example of a typical coordinator dashboard according to an embodiment of the current invention;

FIG. 6 is an example of a patient verification page according to an embodiment of the current invention;

FIG. 7 is an example of appointment time slots in list format according to an embodiment of the current invention;

FIG. 8 is an example of appointment time slots in calendar format according to an embodiment of the current invention;

FIG. 9 is an example of an appointment confirmation window according to an embodiment of the current invention;

FIG. 10 is an example of an active appointment page with timeline according to an embodiment of the current invention;

FIG. 11 is an example of a timeline log according to an embodiment of the current invention;

FIG. 12 is an example of a specify payment window according to an embodiment of the current invention;

FIG. 13 is an example of a send documents window according to an embodiment of the current invention;

FIG. 14 is an example of a select documents window according to an embodiment of the current invention;

FIG. 15 is an example of a downloads window according to an embodiment of the current invention;

FIG. 16 is an example of a single download warning window according to an embodiment of the current invention;

FIG. 17 is an example of a confirm payment window and drop down menu according to an embodiment of the current invention;

FIG. 18 is an example of a provider telemedicine appointment window with video conference dial-in information according to an embodiment of the current invention;

FIG. 19 is an example of a completed appointment window with notes according to an embodiment of the current invention;

FIG. 20 is an example of a cancel appointment location page according to an embodiment of the current invention;

FIG. 21 is an example of a cancelation window according to an embodiment of the current invention;

FIG. 22 is an example of a provider schedule page according to an embodiment of the current invention;

FIG. 23 is an example of a users page according to an embodiment of the current invention;

FIG. 24 is an example of a department creation page according to an embodiment of the current invention;

FIG. 25 is an example of a facility creation page according to an embodiment of the current invention;

FIG. 26 is an example of a patient station creation page according to an embodiment of the current invention;

FIG. 27 is an example of a schedule tab detail page according to an embodiment of the current invention;

FIG. 28 is an example of a schedule creation page according to an embodiment of the current invention;

FIG. 29 is an example of a telemedicine page according to an embodiment of the current invention;

FIG. 30 is an example of a peripheral detail page according to an embodiment of the current invention;

FIG. 31 is an example of a provider station detail page according to an embodiment of the current invention;

FIG. 32 is an example of pool selection page according to an embodiment of the current invention; and

FIG. 33 is an example of a pool edit page according to an embodiment of the current invention.

### DETAILED DESCRIPTION OF THE INVENTION

The current invention now will be described more fully hereinafter with reference to the accompanying drawings, which illustrate embodiments of the invention. This invention may, however, be embodied in many different forms and should not be construed as limited to the illustrated embodiments set forth herein. For example, the present invention is not limited to use in a healthcare environment. Rather, these embodiments are provided so that this disclosure will be thorough and complete, and will fully convey the scope of the invention to those skilled in the art.

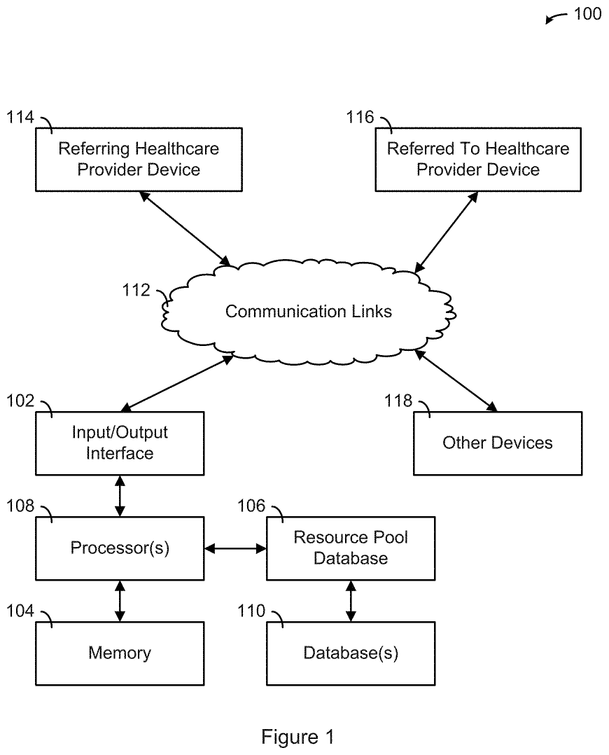

FIG. 1 is a block diagram of a system 100 for scheduling and tracking a telemedicine encounter between a referring healthcare provider and a referred to healthcare provider according to an embodiment of the current invention. The system 100 includes an input/output interface 102, a memory 104, a resource pool database 106, and one or more processors 108 communicably coupled to the input/output interface 102, the memory 104 and the faculty database structure 106. The memory 104 and/or database structure 106 can be local, remote or distributed. Likewise, the one or more processors 108 can be local, remote or distributed. The database structure 106 can be linked to and access data from one or more databases 110, which can be local, remote or distributed. The input/output interface 102 can be any mechanism for facilitating the input and/or output of information (e.g., web-based interface, touchscreen, keyboard, mouse, display, printer, etc.) Moreover, the input/output interface 102 can be a remote device communicably coupled to the one or more processors 108 via one or more communication links 112 (e.g., network(s), cable(s), wireless, satellite, etc.). The one or more communication links 112 can communicably couple the system 100 to referring healthcare provider devices 114, referred to healthcare provider devices 116, and other devices 118 (e.g., databases, remote devices, hospitals, doctors, researchers, patients, etc.). The system 100 can be implemented with various devices, such as, server computers, workstation computers, laptop computers, mobile communications devices, personal data assistants, scanning devices or any other devices capable of performing the functions described herein. Note also that the system 100 may include other components not specifically described herein.

The one or more processors 108 receive a telemedicine scheduling request for a patient from a referring healthcare provider device 114 via the input/output interface 102, the scheduling request includes a patient data and one or more appointment parameters. The scheduling request can include additional data. The one or more processors 108 search the database of resource pool profiles 106 for any medical provider profiles that match the one or more appointment parameters, and provide one or more appointment times that match the one or more appointment parameters to the referring healthcare provider device 114 via the input/output interface 102. The resource pool profiles may include healthcare facility profiles, healthcare department profiles, healthcare unit profiles, healthcare organization profiles, or other profile types. The telemedicine location profiles can include patient stations with video conference capabilities, patient station peripherals for specific patient healthcare evaluations, or other data.

Each appointment time is linked to one or more of the healthcare provider profiles. The appointment times can be linked to other data in the resource pool database 106 or other databases 110. The one or more processors 108 receive a selected appointment time from the one or more appointment times from the referring healthcare provider device 114 via the input/output interface 102, and automatically: (1) send a booking alert to the referring healthcare provider device 114 and a referred to healthcare provider device 116 associated with the selected appointment time via the input/output interface 102; (2) schedule the telemedicine encounter for the healthcare provider profile and the telemedicine location profile at the selected appointment time; and (3) and create an appointment timeline for the patient that includes a set of appointment tasks. In addition, the one or more processors 108 automatically create and provide one or more videoconference links for the telemedicine encounter to the referring healthcare provider device 114 and the referred to healthcare provider device 116 via the input/output interface 102, and automatically track and send alerts upon completion of each of the set of telemedicine encounter tasks.

As used herein, the referring healthcare provider device 114 and the referred to healthcare provider device 116 can be one or more of a computer, a laptop, a handheld device, a mobile device or any other type of device suitable for performing the functionality described herein alone or in conjunction with another device or component, either now or in the future. Note that the referring healthcare provider device 114 and/or the referred to healthcare provider device 116 can be more than one device. The referring healthcare provider and the referred to healthcare provider each can be one or more of a physician, nurse practitioner, physician assistance, nurse, nurse's aid, other healthcare professional, a healthcare coordinator, a healthcare staff or other person.

The one or more appointment parameters can include a patient location, a requested telemedicine location selected from the telemedicine location profiles, a requested medical specialty selected from the one or more medical specialties, a requested medical profile selected from the two or more medical profiles, a requested appointment length, a requested medical provider gender, an appointment urgency, a request for one or more medical peripherals, or a combination thereof. The one or more processors 108 can automatically rank the one or more appointment times based on one or more criteria, which can include a capacity at the two or more telemedicine location profiles, the two or more healthcare provider profiles, a utilization across a set of the resource pool profiles, or any other suitable metric. The one or more processors 108 can provide an assignment of the resource pool profiles in a list or calendar format via the input/output interface 102. Moreover, the system 100 can be integrated into or communicably linked to an electronic medical record (EMR) system.

The one or more processors 108 can assign a role designation to a user, wherein the role designation comprises an institutional administrator, a department administrator, a technical administrator, a presenter, a healthcare provider, or a referring healthcare provider. Moreover, the one or more processors 108 can provide access to the computer system based on a hierarchical structure of the role assignment, an access and a function of the user.

The set of telemedicine encounter tasks can including a payment method, sending a request for a pre-appointment patient medical information, receiving the pre-appointment patient medical information, confirming the payment method, initiating the telemedicine encounter, completing the telemedicine encounter, sending the patient results, receiving the patient results, or other tasks. Accordingly, the one or more processors 108 can perform one or more of the following: receive a payment method from the referring healthcare provider device 114; receive a confirmation of the payment method from the referred to healthcare provider 116; send a request for a pre-appointment patient medical information to the referring healthcare provider device 114; receive the pre-appointment patient medical information from the referring healthcare provider device 114; initiate the telemedicine encounter using the one or more videoconference links; receive a completion of the telemedicine encounter from the referred to healthcare provider device 116; receive the patient results from the referred to healthcare provider device 116; provide the patient results to the referring healthcare provider device 114; or other actions associated with telemedicine encounter or telemedicine encounter tasks.

In addition, the one or more processors 108 can share one or more documents or files between the referring healthcare provider device 114 and the referred to healthcare provider device 116 via the computer system 100. The one or more documents or files can be used for clinical support, administrative support, education, tutoring, training, credentialing of one or more of the resource pool profiles, store and forward telemedicine consultations, or other desired purpose. Moreover, the one or more processors 108 can store, distribute and process the one or more documents for files in the telemedicine encounter or an evaluation of the telemedicine encounter. For example, the one or more processors 108 can receive one or more documents or files from the referring healthcare provider device 114 or the referred to healthcare provider device 116, make the received one or more documents or files available for downloading to the referring healthcare provider device 114 or the referred to healthcare provider device 116, and remove the received one or more documents or files from the computer system after a preset amount of time or until the received one or more documents or files are downloaded.

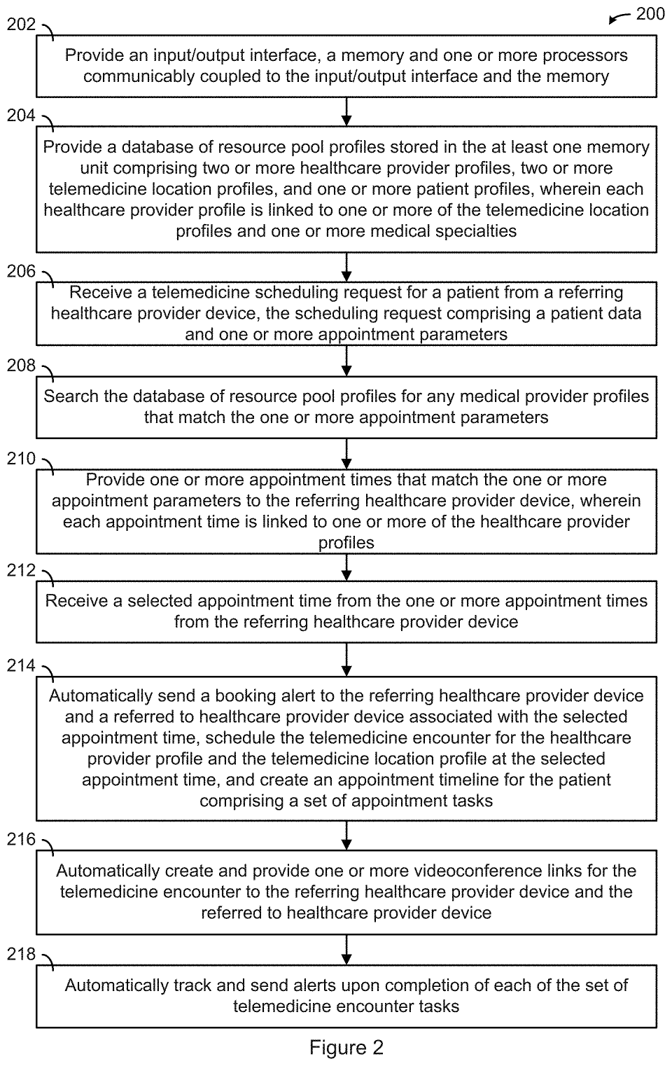

FIG. 2 is a flow chart 200 of a computerized method of scheduling and tracking a telemedicine encounter between a referring healthcare provider and a referred to healthcare provider. An input/output interface, a memory one or more processors communicably coupled to the input/output interface and the memory are provided in block 202. A database of resource pool profiles stored in the at least one memory unit is provided in block 204. The resource pool profiles can include two or more healthcare provider profiles, two or more telemedicine location profiles, and one or more patient profiles, wherein each healthcare provider profile is linked to one or more of the telemedicine location profiles and one or more medical specialties. A telemedicine scheduling request for a patient is received from a referring healthcare provider device in block 206. The scheduling request can include a patient data and one or more appointment parameters. The database of resource pool profiles is searched for any medical provider profiles that match the one or more appointment parameters in block 208. One or more appointment times that match the one or more appointment parameters are provided to the referring healthcare provider device in block 210. Each appointment time is linked to one or more of the healthcare provider profiles. A selected appointment time is received from the one or more appointment times from the referring healthcare provider device in block 212. Automatically in block 214, a booking alert is sent to the referring healthcare provider device and a referred to healthcare provider device associated with the selected appointment time, the telemedicine encounter is scheduled for the healthcare provider profile and the telemedicine location profile at the selected appointment time, and an appointment timeline created for the patient comprising a set of appointment tasks. Automatically in block 216, one or more videoconference links for the telemedicine encounter are created and provided to the referring healthcare provider device and the referred to healthcare provider device. Automatically in block 218, completion of each of the set of telemedicine encounter tasks is tracked and alerts are send upon completion.

In one aspect, the referring healthcare provider device and the referred to healthcare provider device comprise one or more of a computer, a laptop, a handheld device, or a mobile device, and the referring healthcare provider and the referred to healthcare provider each comprise one or more of a physician, nurse practitioner, physician assistance, nurse, nurse's aid, other healthcare professional, a healthcare coordinator or a healthcare staff. In another aspect, the referring healthcare provider device comprises more than one device, or the referred to healthcare provider device comprises more than one device. In another aspect, the resource pool profiles comprise healthcare facility profiles, healthcare department profiles, healthcare unit profiles, or healthcare organization profiles. In anther aspect, the telemedicine location profiles comprise patient stations with video conference capabilities, and patient station peripherals for specific patient healthcare evaluations. In another aspect, method further comprises providing an assignment of the resource pool profiles in a list or calendar format. In another aspect, the computerized system is integrated into or communicably linked to an EMR system.

In another aspect, the method further comprises assigning a role designation to a user, wherein the role designation comprises an institutional administrator, a department administrator, a technical administrator, a presenter, a healthcare provider, or a referring healthcare provider. In another aspect, the method further comprises providing access to the computer system based on a hierarchical structure of the role assignment, an access and a function of the user.

In another aspect, the one or more appointment parameters comprise a patient location, a requested telemedicine location selected from the telemedicine location profiles, a requested medical specialty selected from the one or more medical specialties, a requested medical profile selected from the two or more medical profiles, a requested appointment length, a requested medical provider gender, an appointment urgency, a request for one or more medical peripherals, or a combination thereof. In another aspect, the method further comprises automatically ranking the one or more appointment times based on one or more criteria comprising a capacity at the two or more telemedicine location profiles, the two or more healthcare provider profiles, or a utilization across a set of the resource pool profiles.

In another aspect, the set of telemedicine encounter tasks comprise specifying a payment method, sending a request for a pre-appointment patient medical information, receiving the pre-appointment patient medical information, confirming the payment method, initiating the telemedicine encounter, completing the telemedicine encounter, sending the patient results, or receiving the patient results. In another aspect, the method further comprises one or more of the following: receiving a payment method from the referring healthcare provider device; receiving a confirmation of the payment method from the referred to healthcare provider; sending a request for a pre-appointment patient medical information to the referring healthcare provider device; receiving the pre-appointment patient medical information from the referring healthcare provider device; initiating the telemedicine encounter using the one or more videoconference links; receiving a completion of the telemedicine encounter from the referred to healthcare provider device; receiving the patient results from the referred to healthcare provider device; or providing the patient results to the referring healthcare provider device.

In another aspect, the method further comprises sharing one or more documents or files between the referring healthcare provider device and the referred to healthcare provider device via the computer system. In another aspect, the method further comprises using the one or more documents or files for clinical support, administrative support, education, tutoring, training, credentialing of one or more of the resource pool profiles, or store and forward telemedicine consultations. In another aspect, the method further comprises storing, distributing and processing the one or more documents for files in the telemedicine encounter or an evaluation of the telemedicine encounter. In another aspect, the method further comprises: receiving one or more documents or files from the referring healthcare provider device or the referred to healthcare provider device; making the received one or more documents or files available for downloading to the referring healthcare provider device or the referred to healthcare provider device; and removing the received one or more documents or files from the computer system after a preset amount of time or until the received one or more documents or files are downloaded.

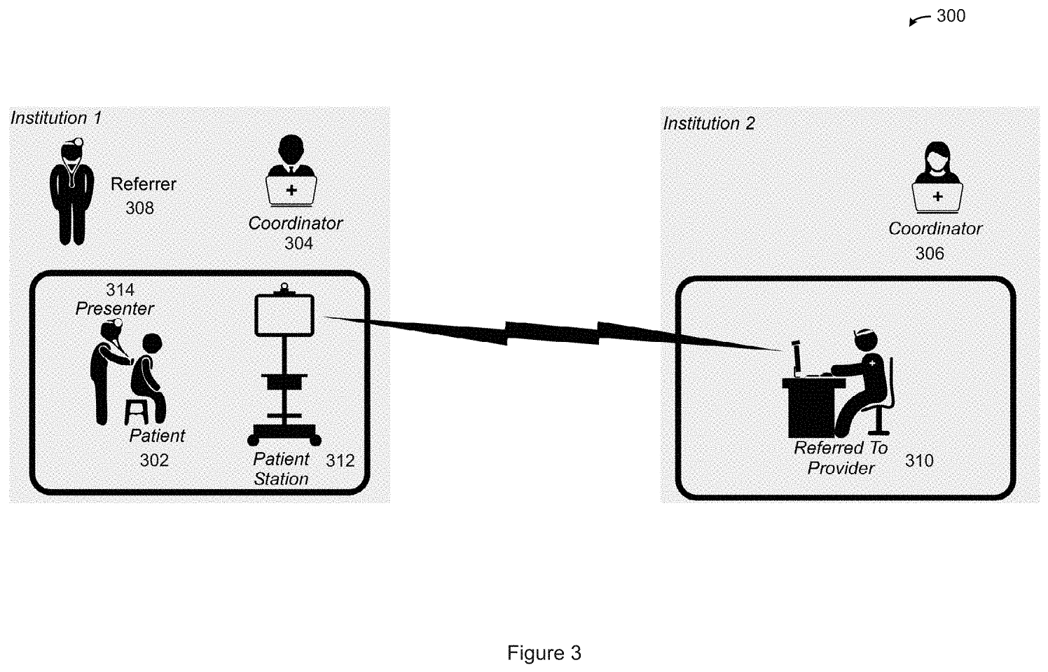

A non-limiting example of the current invention will now be described in reference to FIGS. 3-33. The current invention demonstrates a centralized scheduler and document-sharing platform for intra-enterprise and/or inter-enterprise wide communication, appointment and/or meeting scheduling, and clinical and quality assessment. In one embodiment of the current invention and as illustrated in FIG. 3, the process 300 of scheduling a patient 302 between two different clinical sites and/or healthcare campuses, Institution 1 and Institution 2, is provided with tasks to be completed by staff members or coordinators 304 and 306 at both sides of the clinical consult. In this situation, a referring provider 308 has a patient 302 who needs to be seen by a referred to provider 310. Note that the institutions may or may not be an actual physical facility, but an over-arching body. For example, the University of Texas Medical Branch (UTMB) is an institution, but Angleton Danbury Campus (ADC) ICU is a facility connected to UTMB. The referring provider's coordinator 304 logs into the scheduler and document-sharing platform via a dedicated URL and begins to look for an appointment time. Identifying patient information is entered along with appointment parameters including, but not limited to: zip code of where patient is located (to help find closest telemedicine equipped facility); type of specialty needed or if known, the name of the specialist; length of appointment time; gender of physician desired; urgency of appointment; and/or whether medical peripherals will be needed.

The scheduler and document-sharing platform finds available appointment times. The referring provider's coordinator 304 selects the best appointment time. Email alerts are sent out to the referred to provider 310 and all coordinators 304 and 306. Once the appointment is booked a timeline is created to assist in determining what step happens next until the appointment is complete. Next the referring from provider's coordinator 304 enters in the payment information. Alerts are sent indicating this is complete to the referred to provider's coordinator 306. Then the referring from provider's coordinator 304 uploads patient medical information (PMI) for the consulting provider 310. PMI includes personal health information, generally referring to demographic information, medical histories, test and laboratory results, mental health conditions, insurance information, and other data that a healthcare professional collects to identify an individual and determine appropriate care.

Files only stay on the server for a preset amount of time or until they are downloaded. Next the referred to provider's coordinator 306 downloads the PMI and uploads it into the EMR for the consulting provider 310 to review. The referred to provider's coordinator 306 also verifies payment information. The referred to provider 310 or their coordinator 306 selects initiate telemedicine appointment to see what numbers to dial. Clicking on the links, the videoconference is established between the provider 310 and patient 302 via patient station 312.

The patient 302 is seen via a patient station 312 with the presenter 314 operating the cart and medical peripherals. Afterwards the appointment is marked complete in the scheduler either by the referred to provider 310 or their coordinator 306. The referred to provider 310 creates a report and their coordinator 306 uploads the report into the document-sharing platform. Files only stay on the server for a preset amount of time or until the documents are downloaded. Finally, the referring from provider's coordinator 304 downloads the report and enters it into the patient's EMR file for the referring from provider 308 to review and provide to the patient 302. In another embodiment, the scheduler and document-sharing platform is linked to and can bi-directionally communicate with a plurality of EMR systems, such as EPIC and Allscripts.

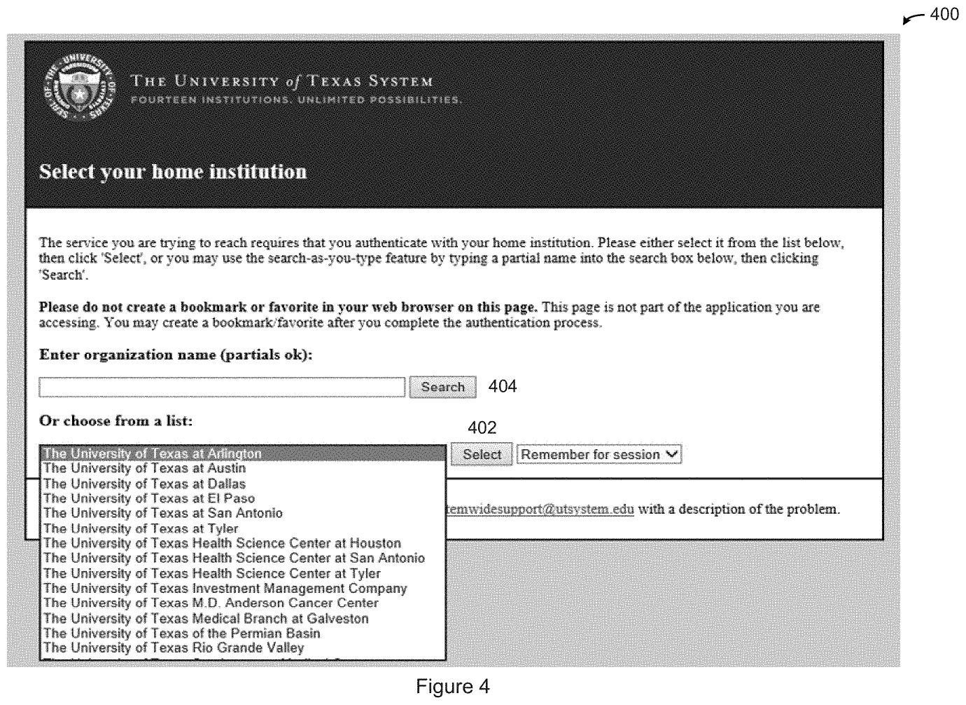

A non-limiting example of the above-described computerized process 300 will now be described in more detail with reference to FIGS. 4-33. The end-user logs onto the scheduler and document-sharing platform via a dedicated URL. The user then clicks on the log in to the scheduler button on the main landing page. This action directs the user to the institutional selection page 400, as shown in FIG. 4. The user clicks on the “Select” button 402 and the system directs the user to the specific institutional login page. The user can also search for an institution using the “Search” button 404. The user will log in using normal institutional credentials and/or SSO credentials. Upon successful log in, the user will be redirected to the user's dashboard page, based on the user's current assigned role.

After logging in, the first page displayed is the user's dashboard. Its content varies based on the role of the user. A first time user has no role, so they will only be able to see users, institutions, facilities, and equipment information.

The user is then able to request role designation and assignment from the system administrator. For example, the user will log into the scheduler and document-sharing platform dedicated URL and then select request access to application. The browser will then display the request to application page. The user then will select the role they need from the drop down list and then select send. Roles available include but are not limited to: institutional administrator, department administrator, technical administrator, presenter, provider, referring provider. Then a message will be sent to the scheduler administrator to complete the role assignment request. In another embodiment, the role assignments will be auto-populated based on preset criteria.

A descriptor showing the user's email, role, and log out link is displayed. In another embodiment, the system automatically logs out the user after a preset amount of time.

A user is able to schedule patient appointments using the scheduler and document-sharing platform. In a further embodiment, appointments can be scheduled intra- and/or inter-enterprise wide to access needed specialty care and equipment that may not be available within the patient's immediate geographical proximity.

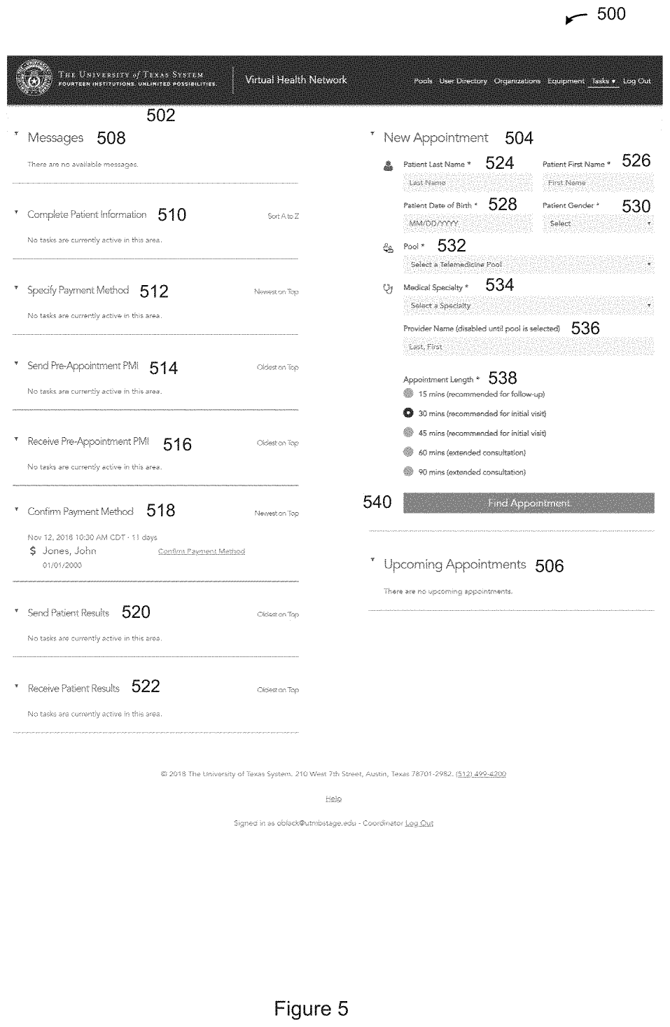

FIG. 5 illustrates an example of a coordinator dashboard 500. The dashboard 500 includes a message and task status section 502, a new appointment section 504 and an upcoming appointments section 506. The message and task status section 502 displays currently available messages and currently active tasks: Messages 508, Complete Patient Information 510, Specify Payment Method 512, Send Pre-Appointment PMI 514, Receive Pre-Appointment PMI 516, Confirm Payment Method 518, Send Patient Results 520, and Receive Patient Results 522. In addition, upcoming appointments are displayed in Upcoming Appointments 506.

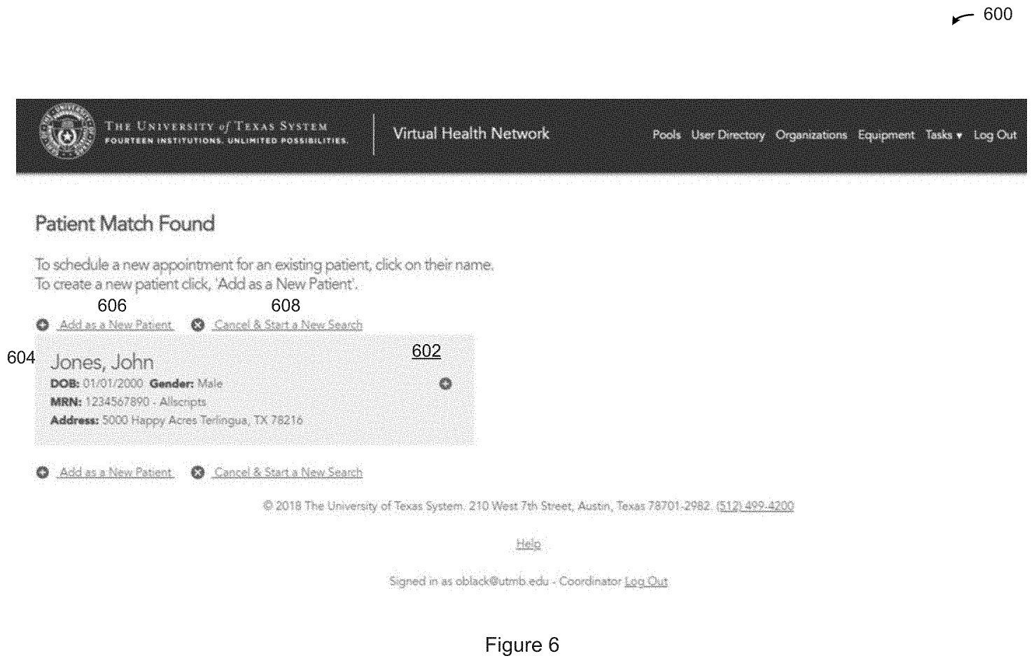

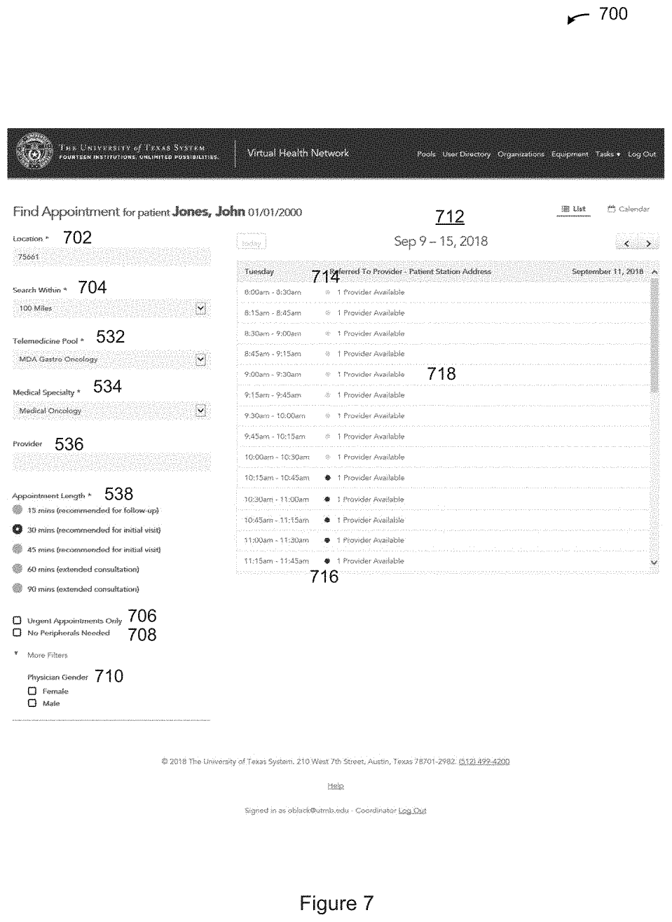

For patient side coordinators, the dashboard 500 is their start for scheduling a patient appointment. The patient side coordinator will input all needed information under the new appointment section 504 of the dashboard page. The information can include patient last name 524, patient first name 526, patient date of birth 528, patient gender 530, pool selection 532, medical specialty selection 534, provider name 536 (disabled until the pool 532 is selected), and appointment length 538 (e.g., 15, 30, 45, 60 or 90 minutes, etc.). In another embodiment, the required information fields within the new appointment section 504 will be auto-populated via direct data pull from an EMR system or other disparate database. After all information is entered, the user will click the find appointment button 540. When selecting a provider, if the provider has multiple specialties, the scheduler and document-sharing platform will ask the user to select the specific specialty for the appointment. The system will first check the patient information to determine if there are any matches in the system. If the patient is a new patient, the next page viewed will be the find appointment page as shown in FIG. 7. If the patient is not a new patient, and currently documented in the system, the patient information 602 will show up on the multiple matching patients' page 600 as shown in FIG. 6. The user will verify the patient by clicking the patient name 604 within the page. To add a new patient, the user clicks the “Add as New Patient” button 606. To cancel and start a new search, the user clicks the “Cancel & Start a New Search” button 608.

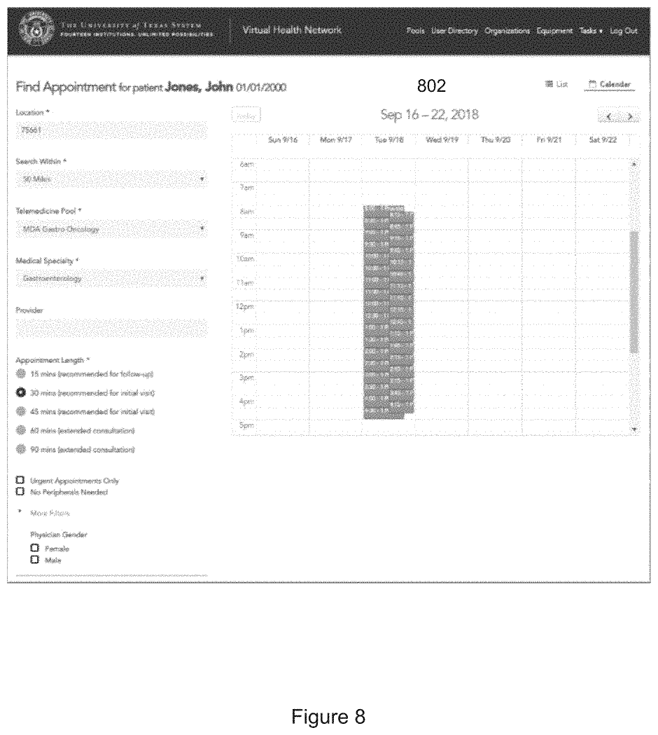

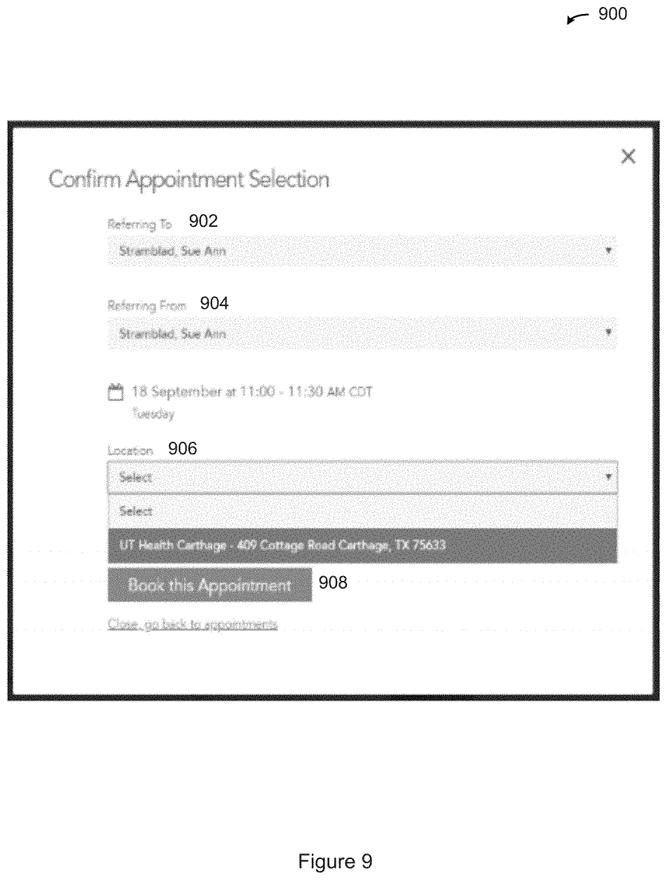

Upon selecting the patient, the user will see the find appointment page 700 highlighting available appointment time slots as illustrated in FIG. 7. The search criteria is displayed to the left as location 702, search within 704, telemedicine pool 532, medical specialty 534, provider 536 and appointment length 538. Additional search filters include urgent appointments only 706, no peripherals needed 708 and physician gender 710. The time slots matching the search criteria can be displayed as a list 712 as shown in FIG. 7 or a calendar view 802 as shown in FIG. 8, both of which include typical navigation buttons. Gray and white highlights 714 signify time slots that are not available. Blue highlights 716 signify available time slots. In some embodiments, unavailable time slots can be hidden so that only the available time slots are displayed. The user then selects the desired appointment time by clicking on the corresponding time slot (e.g., 9:00 am-9:30 am slot 718). After selecting the time slot, a confirmation window 900 will pop up as illustrated in FIG. 9. Using a drop down box, the user selects the referred to provider 902, the referring from provider 904, and the location of the desired appointment 906. Upon verifying information, the user selects book this appointment button 908.

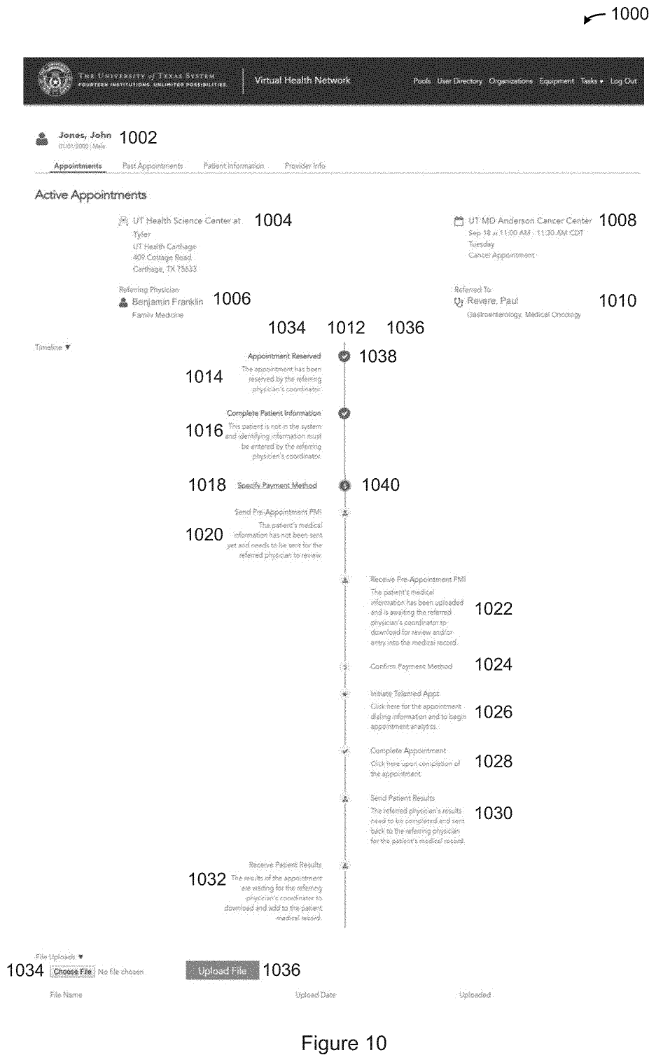

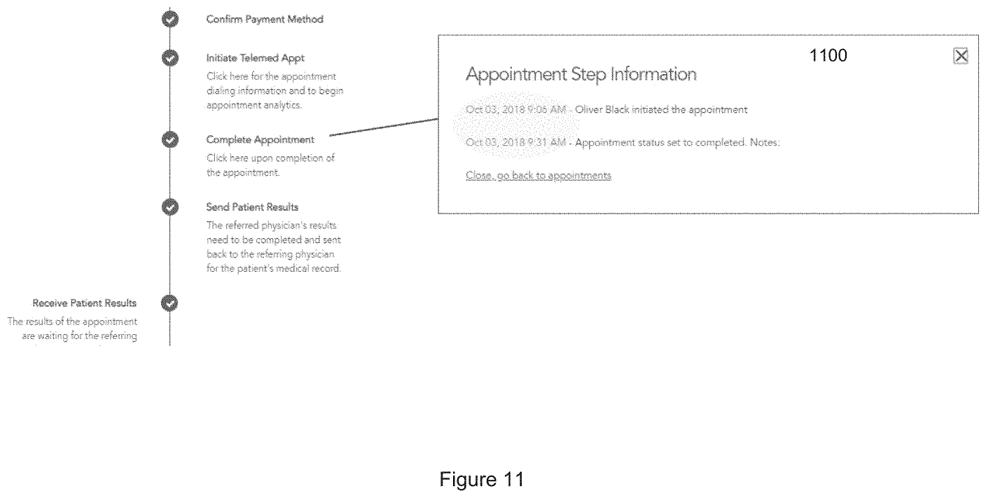

Once the appointment is booked, the web page will redirect to the active appointments page 1000 shown in FIG. 10. In this example, John Jones is the patient 1002, UT Health Tyler is the requesting institution 1004, Benjamin Franklin is the referring physician 1006, UT MD Anderson Cancer Center is the referred to institution 1008, and Paul Revere is the referred to physician 1010. The scheduler and document-sharing platform provides a timeline 1012 outlining a plurality of elements associated with the patient and the patient's appointment. The timeline page shows, step-by-step, the information that is required to be entered into the system and the order in which the information is to be entered (e.g., appointment reserved 1014, complete patient information 1016, specify payment method 1018, send pre-appointment PMI 1020, receive pre-appointment PMI 1022, confirm payment method 1024, initiate telemedicine appointment 1026, complete appointment 1028, send patient results 1030, and receive patient results 1032). The referring providers' personnel 1004 complete all entries 1034 on the left side of the page. All entries 1036 on the right are the responsibility of the referred to providers personnel 1008. Completed entries are signified by a check mark 1038. The entry in blue 1040 signifies the next step to be edited or completed. In some embodiments, the referring provider 1004 is responsible for filling in the specify payment method 1018 as the next step. The timeline 1012 requires the user to complete all entries in sequential order as defined by the system. Users may not arbitrarily skip a step through the timeline 1012. There is a specific order and although once selected some items can be skipped, the process of skipping the item must be verified. Even if an entry has no associated information associated, the user has to click ‘skip’ and the system will log the information regarding, which user and the time when the user, skipped the entry. This sequential entry completion requirement enables all procedures to be followed without mistakenly leaving out any information. As illustrated in FIG. 11, all entry steps are logged with the date, time and who completed that item and any pertinent details as shown in pop-up window 1100. This type of user and activity logging provides an audit trail that can be used to verify compliance with regulatory and/or internal requirements and/or standards. In addition, user and activity logging (e.g., user, date, time, etc.) is not limited to timelines and appointments, but can be implemented on any or all of the systems steps. Clicking on a completed item will create the pop-up window 1100. In embodiment, the timeline 1012 is collapsible and expandable to increase or decrease detail as needed. A file selection 1304 and upload buttons 1036 are displayed at the bottom of the page 1000.

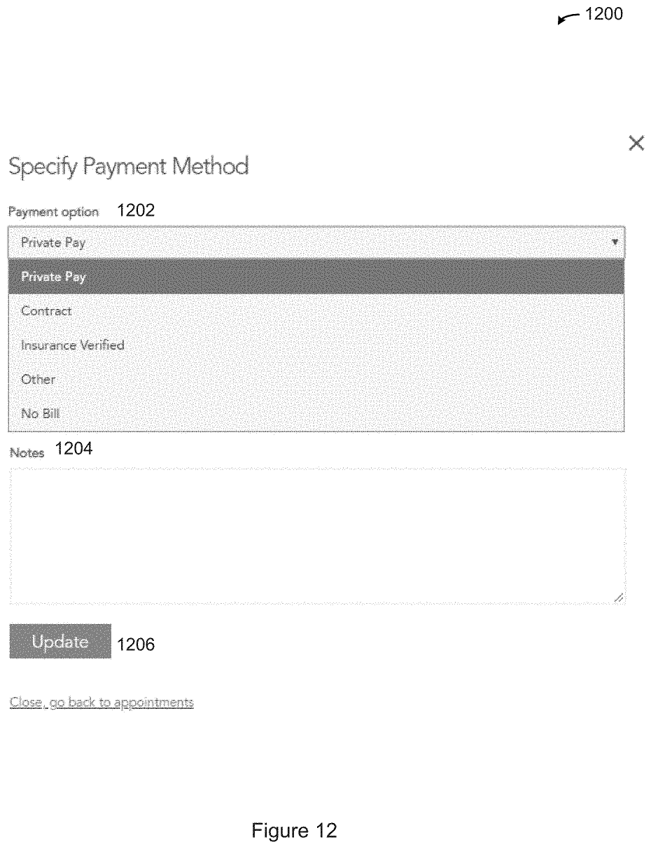

If the patient is new to the scheduler and document-sharing platform, the new patient information is required to be completed. The user fills in additional information including the required information fields highlighted with a red asterisk. The referring provider's staff will select the specify payment method 1018 and a pop-up 1200 will display with payment options 1202 as shown in FIG. 12. The user will select the appropriate payment method (e.g., private pay, contract, insurance verified, other, no bill, etc.). There is a notes section 1204 for adding additional information including but not limited to contract number and insurance number. Once the new information is added, the user will select the update button 1206 to update the records and refresh the page.

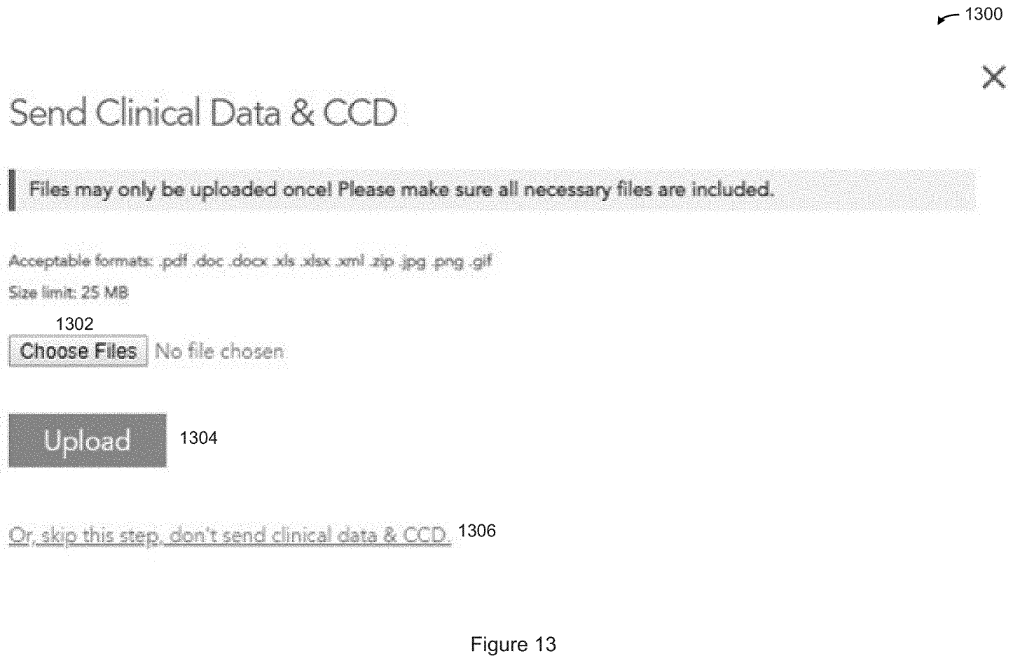

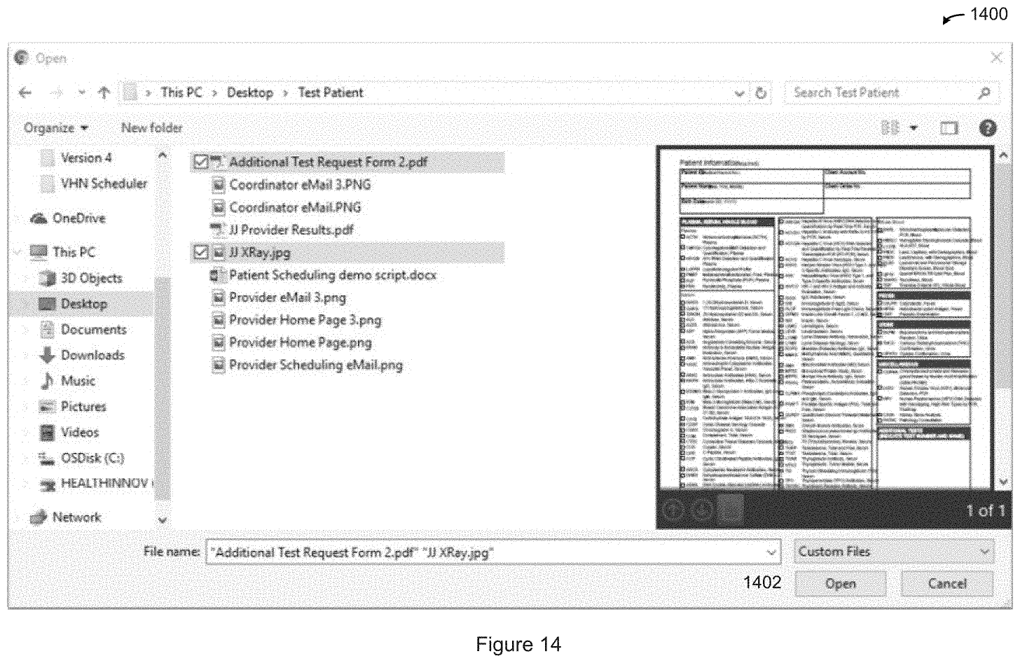

Pre-appointment PMI information can be sent to and from participating institutions, facilities, and/or providers. FIG. 13 illustrates a pop-up 1300 that is displayed and allows pre-appointment PMI information to be uploaded and sent when send pre-appointment PMI 1020 is selected in FIG. 10. If the coordinator has patient information in electronic format, it may be uploaded to the system to send to the referring to provider for insertion in the EMR. Acceptable formats for documents include, but are not limited to, pdf, doc, docx, xls, xml, zip, jpg, MP3, MP4, and/or gif. Any document size restrictions and/or configuration requirements may be preset. The files are selected using the choose files button 1302, and uploaded to the system using the upload button 1304. These files will be available on the server for a preset amount of time or until they are downloaded. FIG. 14 illustrates a document file structure 1400 to be selected and sent. Multiple documents can be selected and sent at once buy the user holding down the “CTRL” button and clicking the individual files within the file structure. When all desired files are selected, the user will select the open button 1402 to return to the send clinical data dialog box. When the files are ready to be sent, the user will press upload 1304 to send the files as shown in FIG. 13. If the coordinator has no files to send, the coordinator may select the skip this step, don't send clinical data and CCD link 1306. The scheduler and document-sharing platform will ask for the confirmation that no files are to be sent. Upon confirmation, the send pre-appointment PMI 1020 shown in FIG. 10 will be marked complete and will be inaccessible to further edit. If additional files need to be sent, the user can utilize the file uploads button 1036 at the bottom of the timeline page 1000.

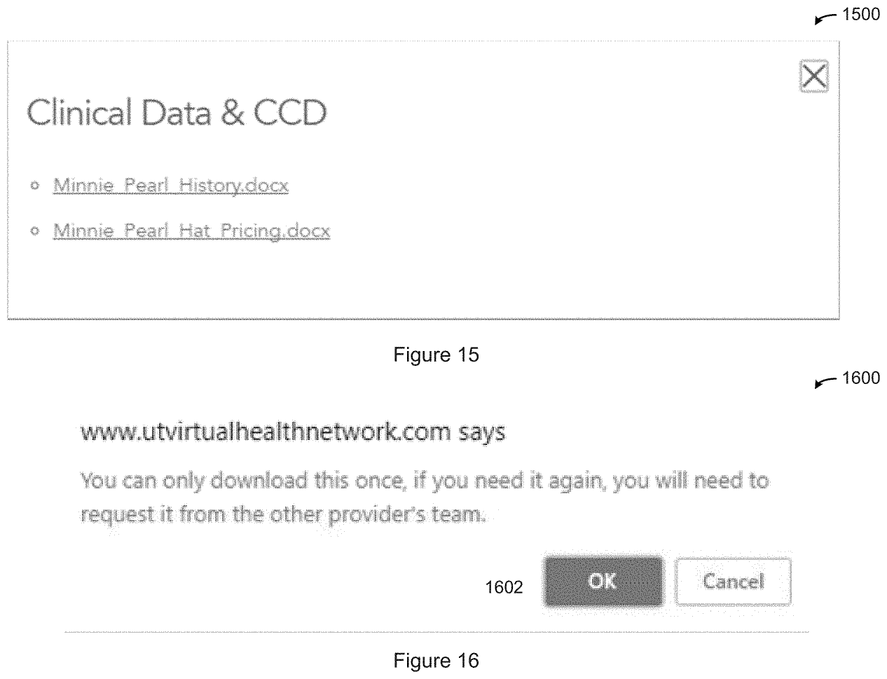

If patient data is uploaded, then the referred to provider's staff will need to download the data and/or documents for entry and/or upload in their EMR. In one embodiment, this data and/or document upload is an automatic process. As illustrated in FIG. 15, a pop-up window 1500 will appear to allow the user to select the documents to download. The user will click on each item to download. As illustrated in FIG. 16, a pop-up 1600 will alert the user that each item can be downloaded once. The user will click OK 1602 and the user will be prompted to save the download. In one embodiment, files can be encrypted in order to maximize adherence to HIPPA regulations.

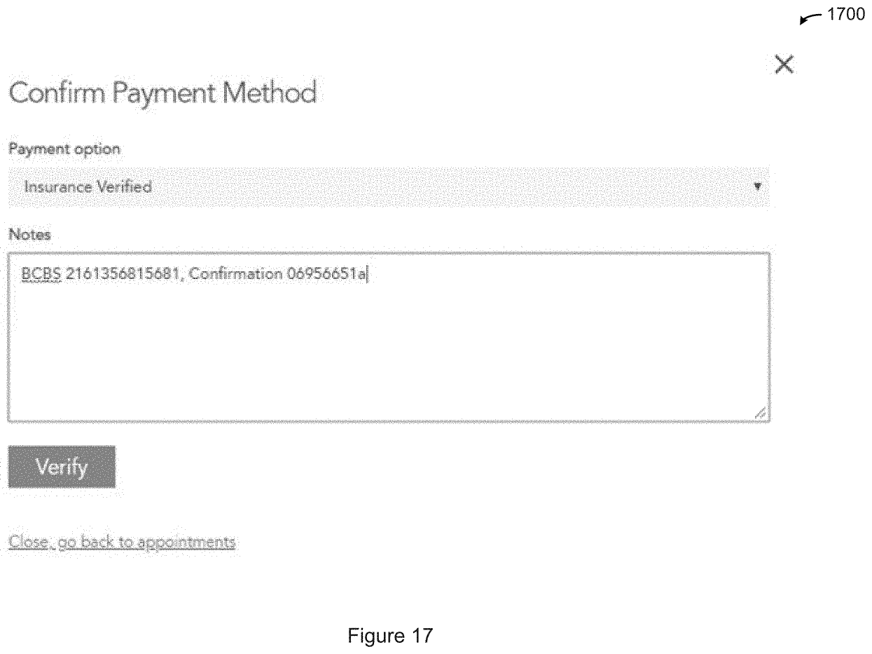

At this point, the referred to provider's staff needs to confirm payment information. This confirmation is to verify that the services to be rendered will be compensated either by the patient, the provider, or the payer. FIG. 17 illustrates the confirm payment method pop-up 1700. By selecting the confirm payment method 1024 in FIG. 10, a pop-up 1700 appears to read the payment information entered by the referring from provider's staff. Once the payment information is confirmed by clicking the verify button 1702, the telemedicine appointment can be initiated.

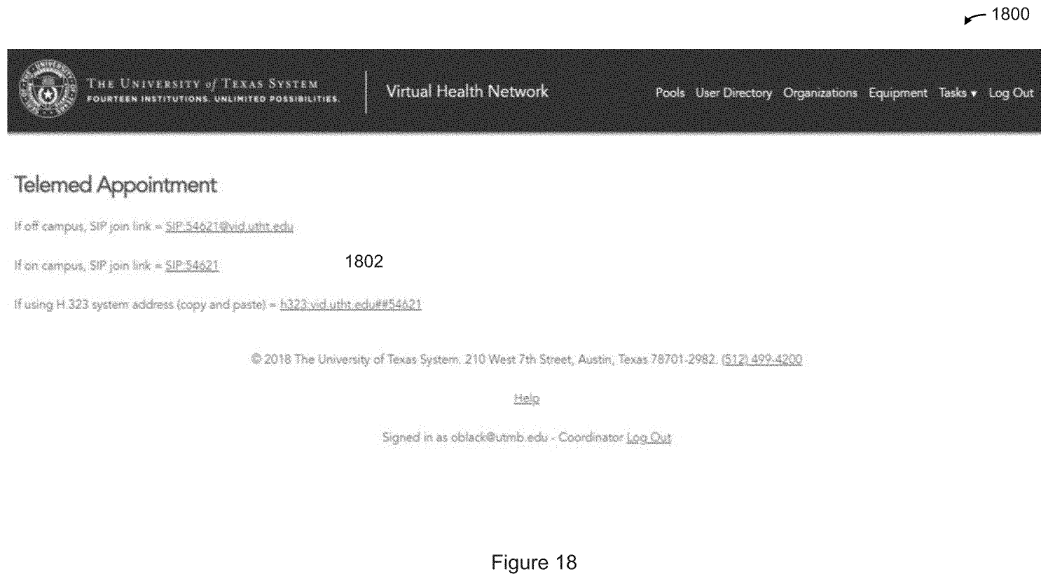

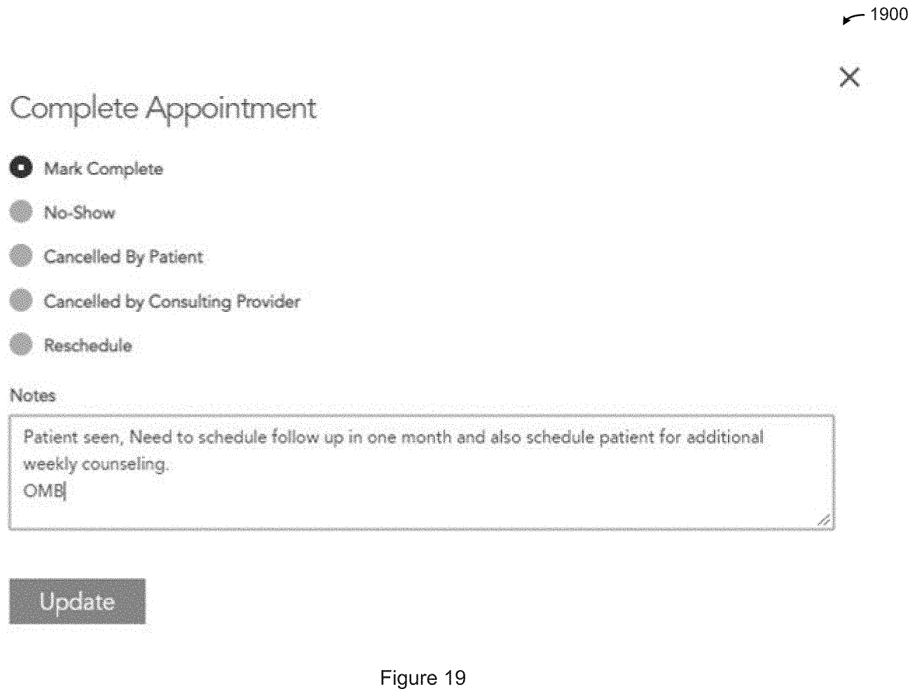

At appointment time, the referred to provider will initiate the telemedicine appointment by selecting the corresponding button 1026 in FIG. 10 that will open a page 1800 as shown in FIG. 18 with the video dialing information for the provider to contact the patient site. If using a workstation and compatible video conferencing software, clicking on the appropriate link 1802 will launch the software and dial the call. At this point, the patient and the providers conduct the clinical aspects of the appointment via the videoconference and use of associated peripherals. In one embodiment, all clinical data is collected and uploaded to the appropriate database and/or system, including but not limited to, an EMR. A timeline link ending the appointment and recording the time used for seeing the patient can be displayed. Selecting the complete appointment button 1028 in FIG. 10 opens a pop-up 1900 as shown in FIG. 19, which allows the appointment to be marked complete, no-show, cancelled by patient, cancelled by consulting provider or reschedule, and allows notes to be entered. A pop-up allowing the user to provide satisfaction scoring of the clinical encounter can also be displayed. In addition, a notes field for both clinical and quality aspects of the appointment and clinical encounter can be displayed. In one embodiment, the satisfaction scoring methods can be used to assess a plurality of clinical and non-clinical quality metrics.

If the referred to provider has a report or other patient information in electronic format, it may be uploaded to the system to send to the referred from provider for insertion in the corresponding EMR. In one embodiment, this data and/or document transfer is automatic. If patient data (results form the consult) are uploaded, then the patient's coordinator may need to download the data and/or documents in to the local EMR. Users may send additional files at any time in the appointment process by using the file upload section located at the bottom of the timeline.

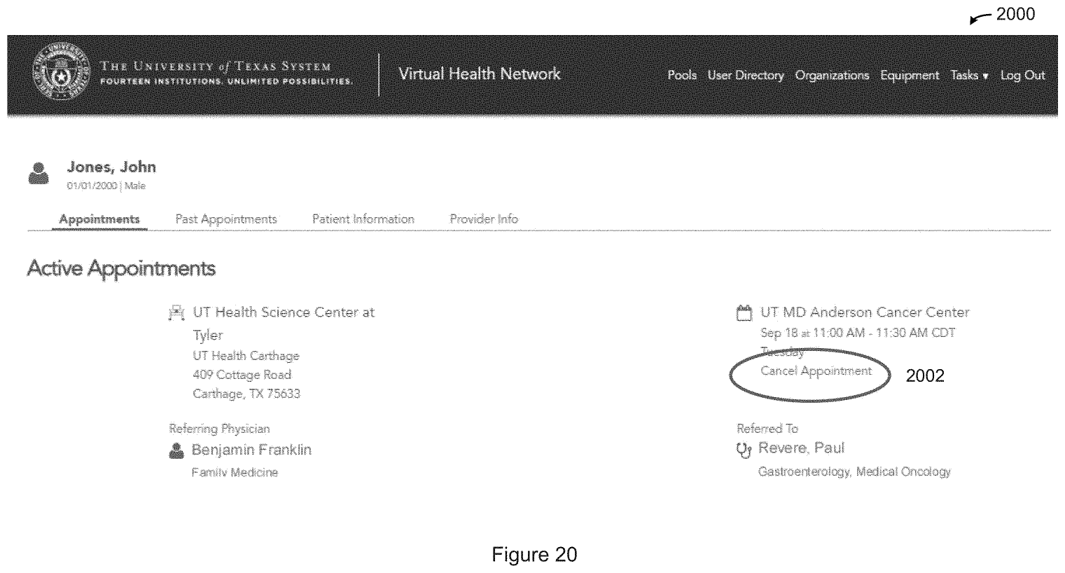

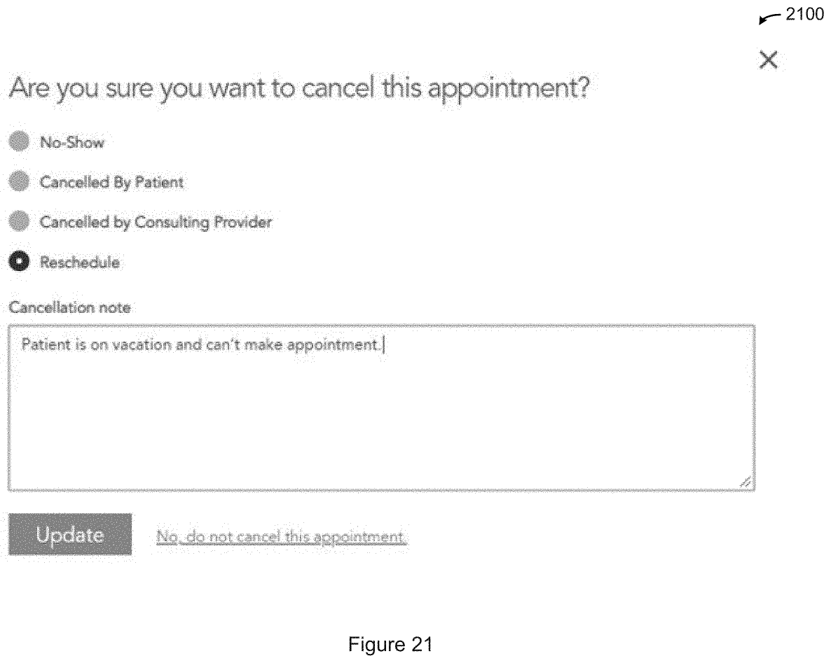

Users may cancel an appointment at any time prior to an appointment taking place using the page 2000 shown in FIG. 20. The user can select cancel appointment 2002 under the active appointments for the patient. As illustrated in FIG. 21, a pop-up 2100 will be displayed and the user will select the reason for the cancellation (e.g., no-show, cancelled by patient, cancelled by consulting provider, reschedule, etc.) and add notes if desired.

The embodiments of the current invention outlined above cover the processes by which the clinical and non-clinical staff across the enterprise engage and coordinate activities to schedule appointments, conduct clinical engagements, and report, record, and share the result of the clinical engagements. In order for this process to be possible, an infrastructure consisting of several interconnected, institutions, departments, facilities, equipment and people are required. It is the role of the institutional administrators and departmental administrators to create this infrastructure within the scheduler and document-sharing platform. In some embodiments, administrators will create departments, facilities, patient stations, and peripheral equipment. These administrators are also responsible for role assignments within the scheduler and document-sharing platform.

For the purposes of assigning roles within the system, institutional administrators will see access requests on their home page. The institutional administrators will click on the desired users pencil icon to respond to the request. The selected user's information page will then be displayed. They will click user role from the drop down menu and select the desired functionality of the user. At this point, the administrator may edit or add user information including but not limited too name, phone number, and address. Users must be assigned an institution by clicking on add an institution and highlighting the desired institution affiliation. Depending on role, users may also need to be assigned a connection to a department and facility. Typically, a department is within an institution and has at least one, if not multiple facilities connected to it. A facility is the physical location where a provider, telemedicine cart presenter is physically located. It is possible to have multiple facilities within one building but typically they have different room numbers, phone numbers, etc.

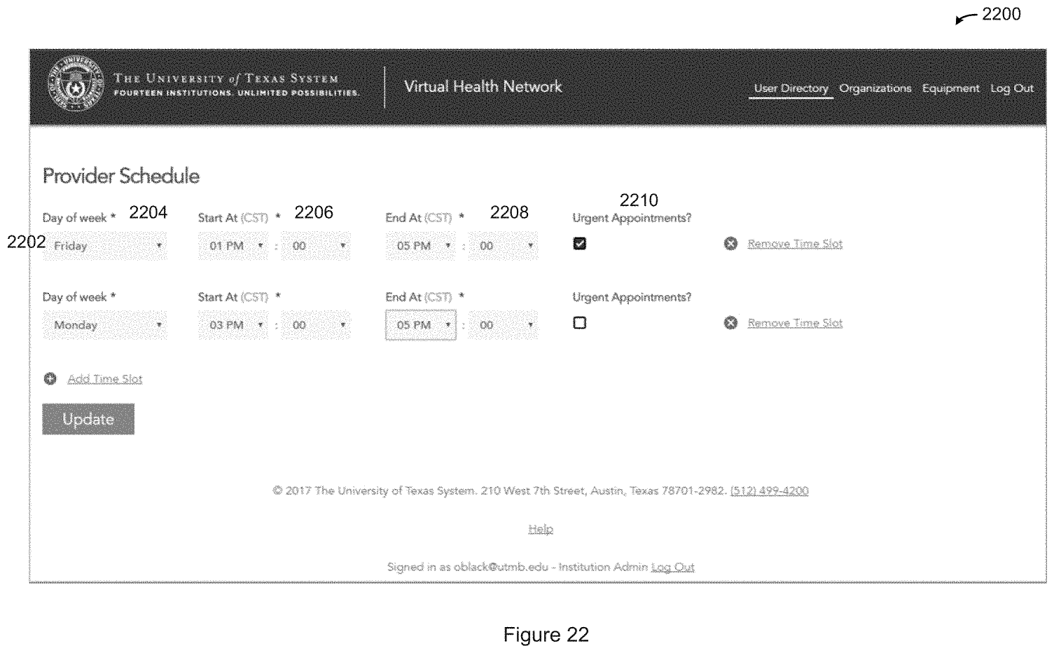

If the provider's available time schedule is known, select the tab to add time when the provider is available for telemedicine appointments. FIG. 22 illustrates embodiments where provider schedules can be updated via page 2200. For each segment of time available 2202, the user will select the day of week 2204, start at 2206, and end at times 2208 as well as if they accept urgent appointments 2210. Additional time slots can be added as needed and the changes are immediate and available. In one embodiment, the user is automatically notified of any role changes.

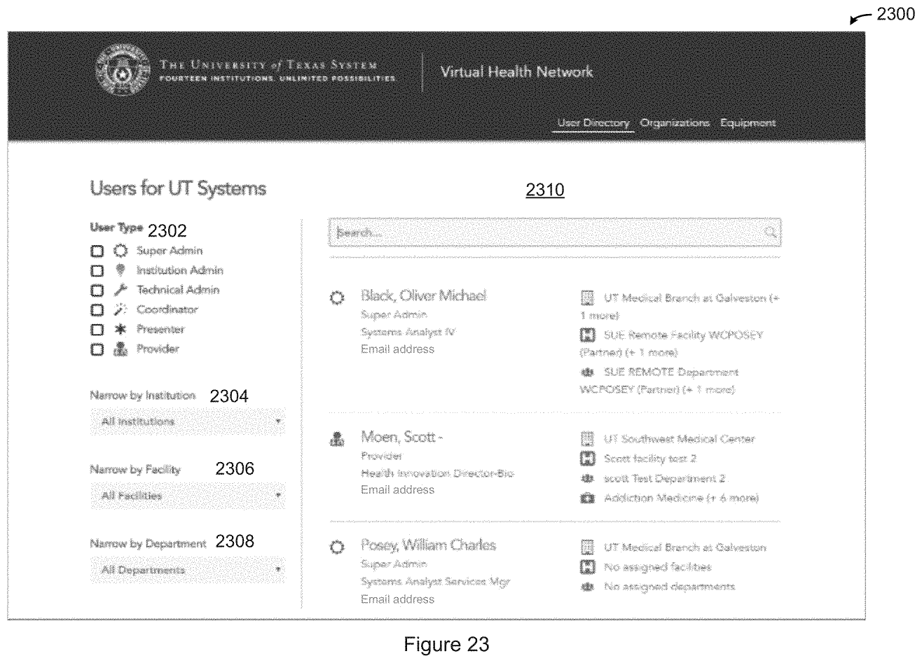

Existing user roles can be edited by selecting user directory 2300 as illustrated in FIG. 23. If the type of user is known, there are filters to sort by roles 2302, institution 2304, faculty 2306 and department 2308. The user can be selected by scrolling down and selecting the specific user to edit or a search can be performed on the right portion 2310 of the page 2300. The user's information page will be displayed and the administrator may change information, roles, and/or connections. If a user leaves the institution their login rights will be revoked by the institution's active directory however their personal data may still be in the scheduler and document-sharing platform. The data can be removed manually by changing the user role to staff. Likewise, if a user stays within the institution, but no longer needs to have an active role in the platform, their role can be changed to staff. In one embodiment, the role changes for existing or non-active employees are automatic.

The system provides for the assignment of one or more role designations to users. The role designations may include, but are not limited to:

Coordinator—a coordinator schedules and coordinates patients at one institution and providers at another institution together to create an appointment. Coordinator roles are available on both the patient's institution side and the provider's institution side. Coordinators can do the following: create an appointment, input patient information, input or verify payment information, upload patient medical information, download patient medical information, and/or receives generic emails concerning updates to appointments.

Departmental Administrator—can create/edit users, departments, facilities and pools associated with their department.

Institutional Administrator—can create/edit users, departments, facilities and pools associated with their institution.

Presenter—This user is the person who “presents” the patient to the referred to provider during the telemedicine session.

Referred To Provider—a provider with the special skills desired for a consult. A provider can do all the items a coordinator can do as well as they receive appointment updates with calendar events to allow them to be easily added to their calendars for notification when an appointment is about to occur. Referred To Providers may have patients referred to them for a telemedicine consultation and they may also refer patients to others to be seen.

Referring Only Provider—a provider with the patient who needs to see a specialist (referred to provider) for a consult. The referring healthcare provider can complete all tasks of a coordinator. In this role designation, they can only refer a patient to other providers for a consultation and cannot have patients referred to them.

Staff—Can view only. No active role in the scheduler. Users are assigned this role when leaving their role.

Super Administrator—This type of user can do anything. This role can only be assigned by other super administrators.

Technical Administrator—Can create/edit telemedicine equipment including patient stations, telemedicine rooms and peripherals.

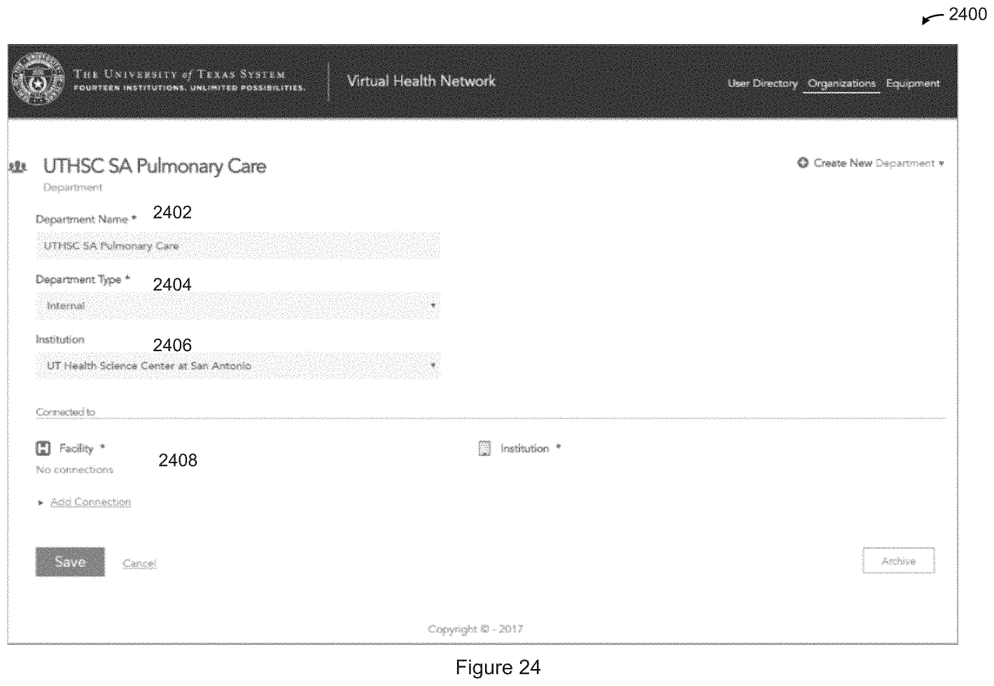

Administrators have the ability to design department and facility structures within their specific institution profile. In an embodiment and illustrated in FIG. 24, users can create new departments under create new department page 2400. The user must provide the department name 2402, department type 2404, institution name 2406, and affiliate connections 2408. The department type can either be internal (a department within the institution) or partner (a department within a partnering organization). Edits and deletion of existing departments are also possible.

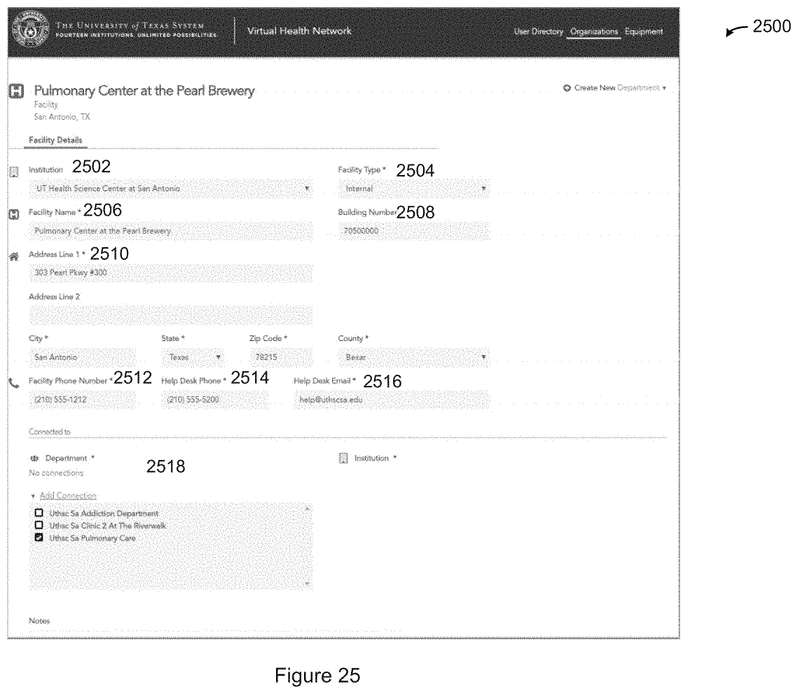

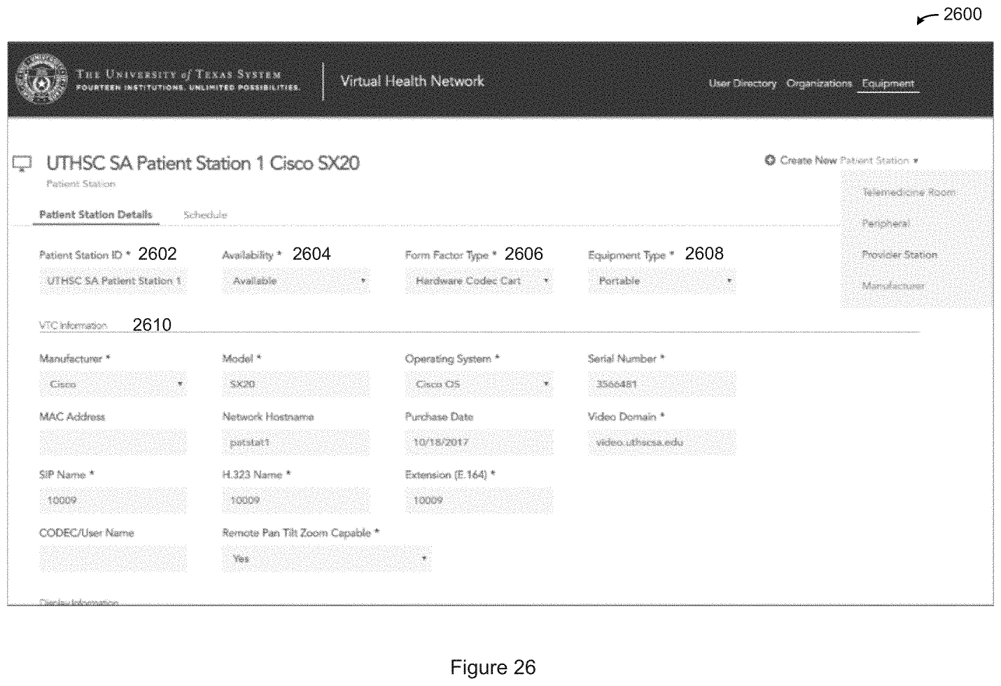

Administrators can also add, edit, and delete facilities within their institution profile. As outlined in FIG. 25, new facility registrations 2500 require institution name 2502, facility type 2504, facility name 2506, building ID 2508, building address 2510, phone number 2512, help desk phone number 2514, help desk email 2516, and affiliate structure 2518. Facility type can be either internal (facility within the institution) or partner (a facility within a partnering organization). In one embodiment, institution and facility specific equipment, such as telemedicine carts, with peripherals, can be registered and tracked for assignment and utilization as shown in FIG. 26. Page 2600 illustrates the equipment details for a patient station. The equipment registration includes, but is not limited to: patient station ID 2602, availability 2604, form factor type 2606, equipment type 2608, VTC information 2610, and display information.

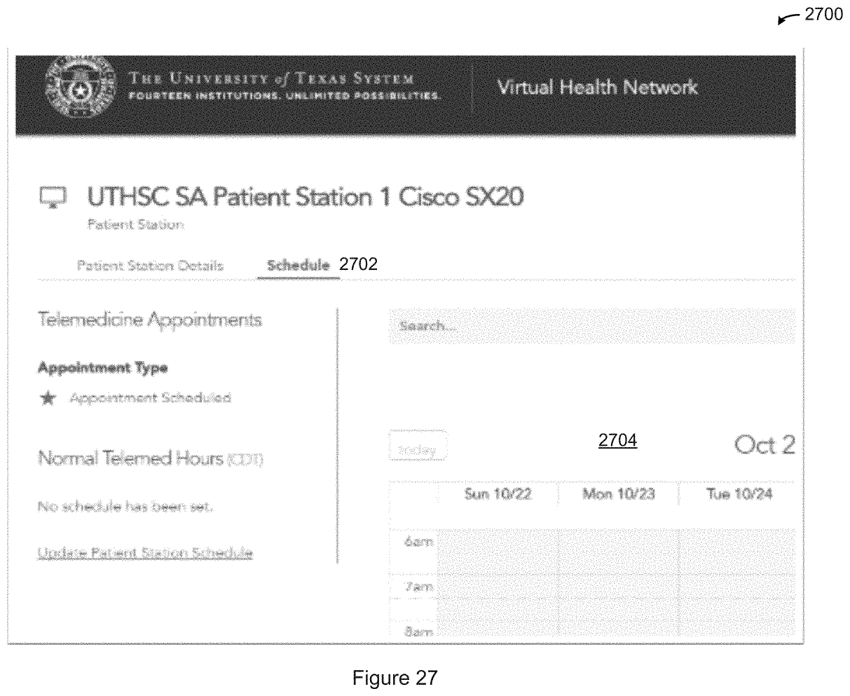

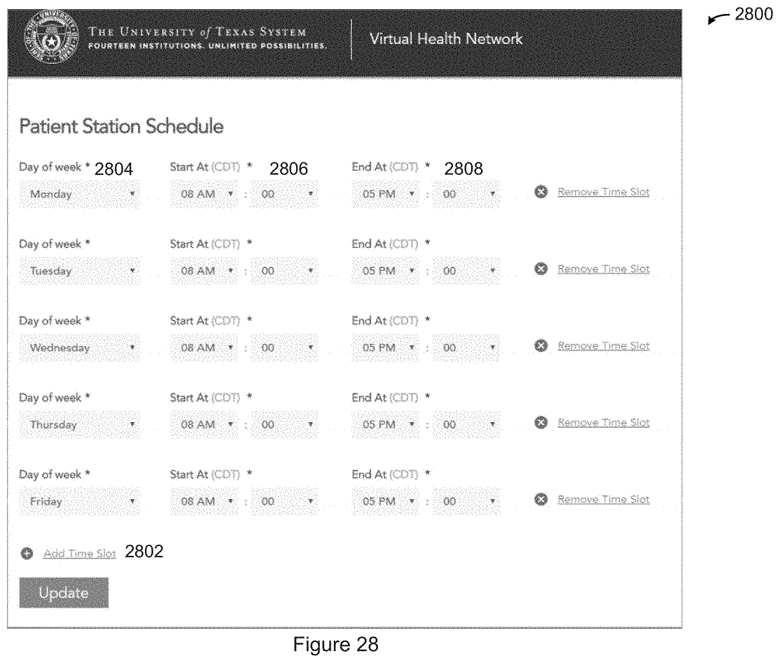

Equipment used within the scheduler and document-sharing platform can be booked for appointment specific clinical engagements after the equipment has been registered within the platform. FIGS. 27 and 28 illustrate the patient station equipment scheduling. In order to book the equipment, the user will click the schedule tab 2702 at the top of the patient station page 2700. On the patient station schedule 2800 shown in FIG. 28, the user will select add time slot 2802. The user will set the day of week 2804, start 2806 and stop 2808 times for availability. In one embodiment, the equipment schedule will be shown in a calendar 2704 and/or list format 2800.

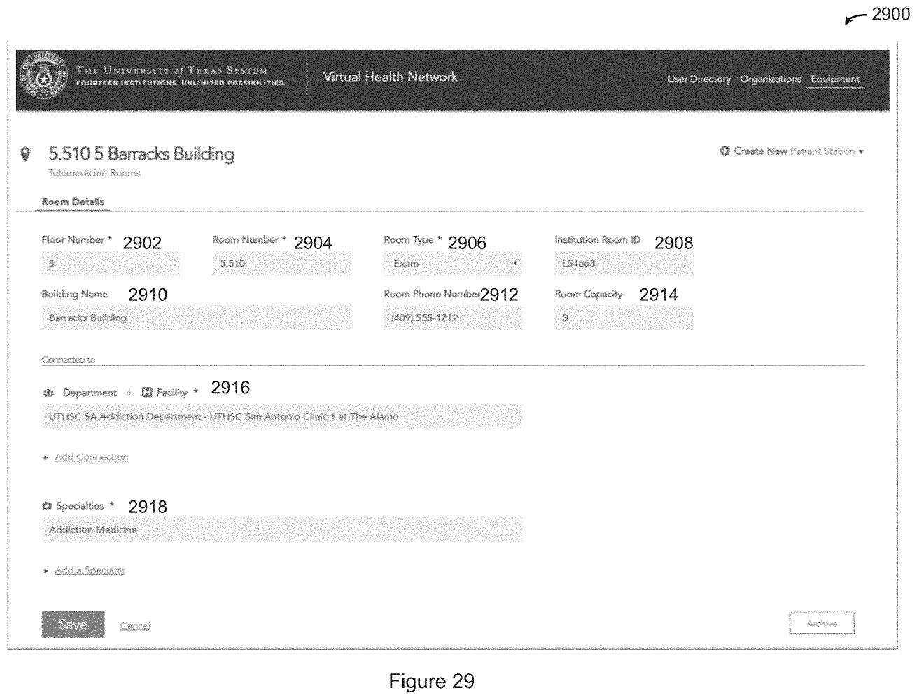

In addition to booking the new patient station with appropriate peripherals, the user will need to book a room within the facility for use during the telemedicine encounter (referred to as a “telemedicine room”) using page 2900 shown in FIG. 29. To register a room within the scheduler and document-sharing platform, the user will need to provide the following information: floor number 2902, room number 2904, room type 2906, institution room ID 2908, building name 2910, room phone number 2912, room capacity 2914, affiliate structure 2916, and specialty type 2918.

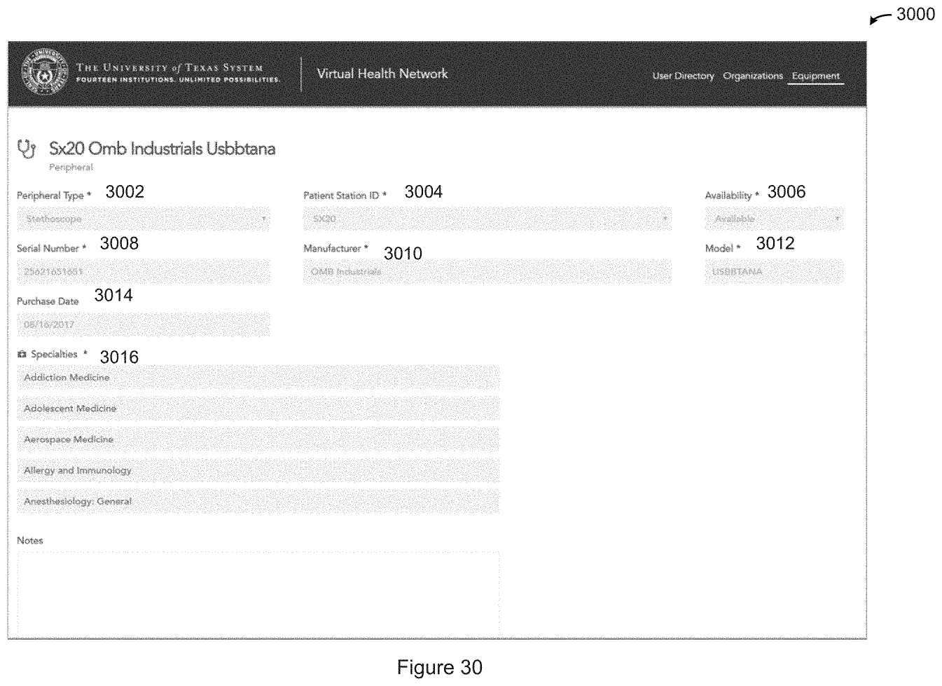

In addition to patient station equipment and rooms, the user can register specific peripherals to be added to a booked patient station using page 3000 shown in FIG. 30. The peripherals are a medical device assigned to a patient station and will be specific to the type of telemedicine encounter. For example, a blood pressure cuff and stethoscope may be used for general examinations while an otoscope may only be needed for an ENT examination. The peripheral registration includes but is not limited to: peripheral type 3002, patient station ID 3004, availability 3006, manufacturer 3008, model 3010, serial number 3012, purchase date 3014, and specialties 3016.

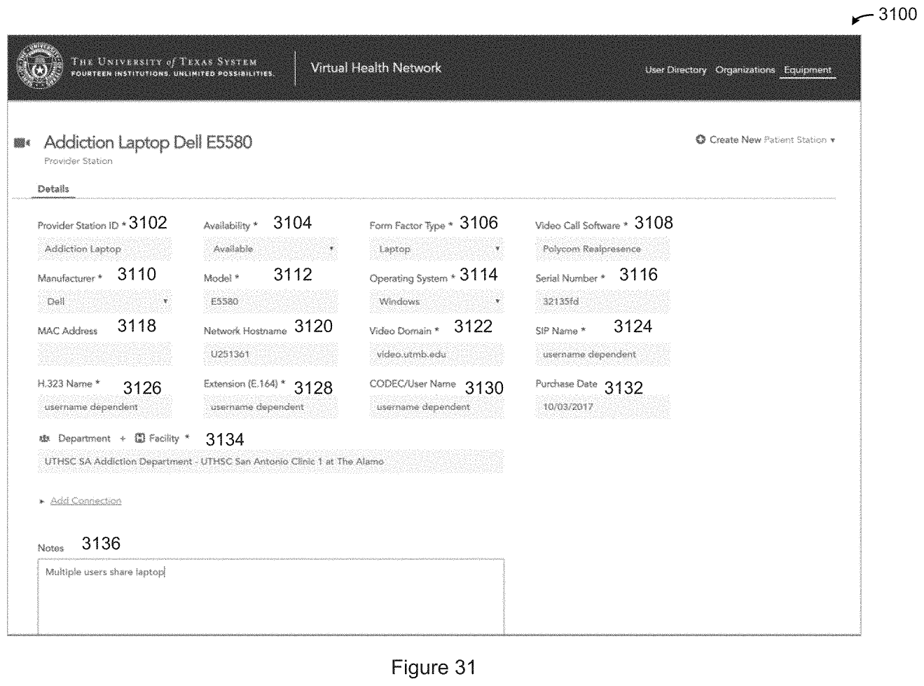

In addition to the patient work station that is required at the patient side of the clinical consult, the providing clinician at the remote site must also have access to communication equipment to conduct the clinical engagement via telemedicine. The provider station, as it is known for the purposes of the current invention, must also be registered and tracked. The provider station is a system the consulting provider uses to connect with video and audio to a patient station for a consult (e.g., a laptop with a speaker/mic pick up and a set of headphones, etc.). FIG. 31 illustrates the registration details of the provider station. The provider station registration 3100 includes but is not limited to: provider station ID 3102, availability 3104, form factor type 3106, video call software 3108, manufacturer 3110, model 3112, operating system 3114, serial number 3116, MAC address 3118, network hostname 3120, video domain 3122, SIP name 3124, H.323 name 3126, extension 3128, CODEC/username 3130, purchase date 3132, affiliate connection 3134 and notes 3136.

Once all institutions, facilities, equipment, and rooms have been registered and made available through the scheduler and document-sharing platform, the process and infrastructure are in place for the enterprise to schedule clinical encounters around a plurality of specific clinical specialties including but not limited to oncology, infectious disease, rheumatology, and nephrology. In one embodiment, the scheduling permissions of users on the scheduler and docu n sharing platform are controlled by a mechanism called a telemedicine pool. A telemedicine pool is comprised of providers, coordinators, and patient stations. In other words, telemedicine pools are groups made up of users (providers, coordinators, etc.) and facilities that are all related through the membership in the pool to allow them to book appointments with other members and facilities in that pool. When a coordinator has been added to a pool, they have permissions to schedule an appointment with any provider that is within that pool. The ability to create pools, and add or remove items from those pools, is restricted to administrative users.

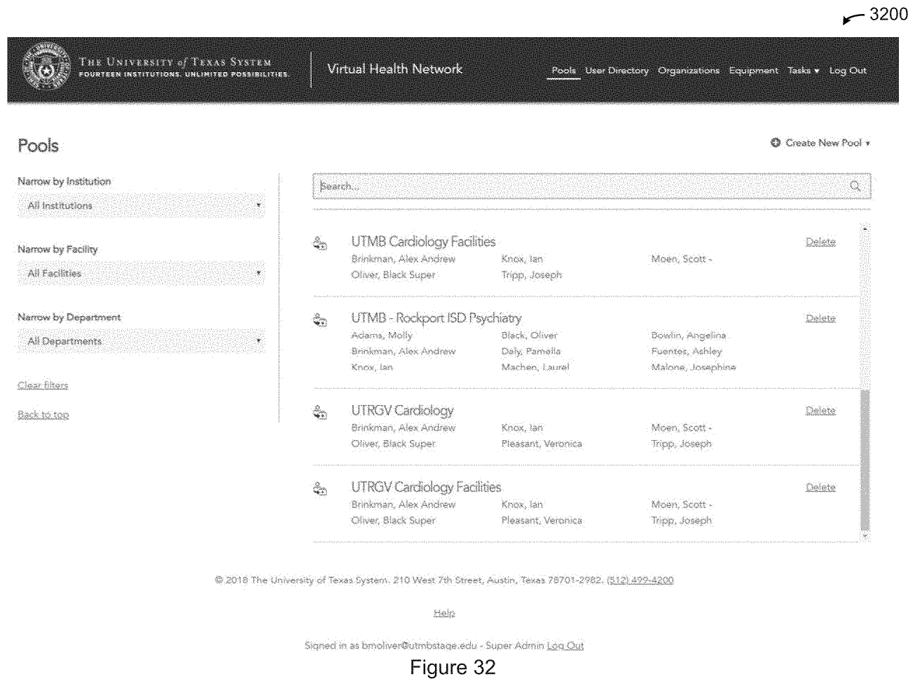

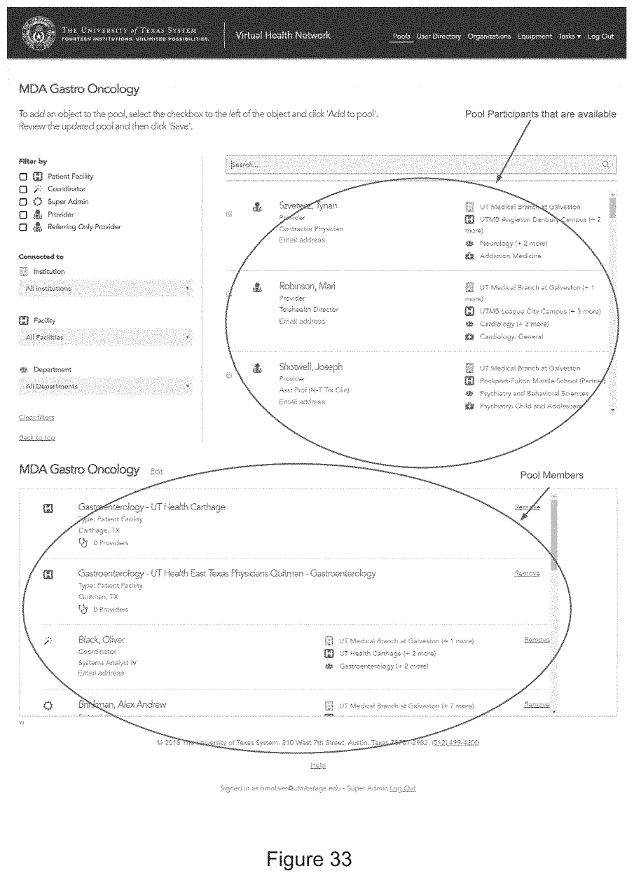

The appointment search algorithm will return an available appointment for unique time slot that meets the following criteria: 1) the provider has availability in their schedule, 2) the provider's medical specialty matches the specialty specified in the search, and 3) The patient station has availability in its schedule where the provider and patient station are within the pool specified in the search. FIGS. 32 and 33 illustrate examples of the specialty specific pools. This construct allows pools and members to utilize the scheduler and documentation system to achieve maximum utilization and assignment efficiencies.

In addition to clinical engagement documents, the scheduler and document-sharing platform has store and forward capabilities. This embodiment allows for patients and clinicians to access information and tutorial materials directly related to non-acute clinical conditions. For example, a primary care physician could access dietary information to review with a patient without having to engage a dietitian in real-time. In another embodiment, credentialing documents are stored, edited, and transmitted to facilitate and fast track clinician credentialing requirements intra- and inter-enterprise wide.

To facilitate the understanding of this invention, a number of terms are defined below. Terms defined herein have meanings as commonly understood by a person of ordinary skill in the areas relevant to the present invention. Note that these terms may be used interchangeably without limiting the scope of the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

All of the systems, devices, computer programs, compositions and/or methods disclosed and claimed herein can be made and executed without undue experimentation in light of the present disclosure. While the systems, devices, computer programs, compositions and methods of this invention have been described in terms of preferred embodiments, it will be apparent to those of skill in the art that variations may be applied to the systems, devices, computer programs, compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A computerized method of scheduling and tracking a telemedicine encounter between a referring healthcare provider and a referred to healthcare provider, comprising:
providing an input/output interface, a memory, one or more processors communicably coupled to the input/output interface and the memory;
providing a database of resource pool profiles stored in the at least one memory unit comprising two or more healthcare provider profiles, two or more telemedicine location profiles, and one or more patient profiles, wherein each healthcare provider profile is linked to one or more of the telemedicine location profiles and one or more medical specialties;
receiving a telemedicine scheduling request for a patient from a referring healthcare provider device, the scheduling request comprising a patient data and one or more appointment parameters;
searching the database of resource pool profiles for any medical provider profiles that match the one or more appointment parameters;
providing one or more appointment times that match the one or more appointment parameters to the referring healthcare provider device, wherein each appointment time is linked to one or more of the healthcare provider profiles;
receiving a selected appointment time from the one or more appointment times from the referring healthcare provider device;
automatically sending a booking alert to the referring healthcare provider device and a referred to healthcare provider device associated with the selected appointment time, scheduling the telemedicine encounter for the healthcare provider profile and the telemedicine location profile at the selected appointment time, and creating an appointment timeline for the patient comprising a set of appointment tasks;
automatically creating and providing one or more videoconference links for the telemedicine encounter to the referring healthcare provider device and the referred to healthcare provider device; and
automatically tracking and sending alerts upon completion of each of the set of telemedicine encounter tasks.

2. The method of claim 1, wherein the one or more appointment parameters comprise a patient location, a requested telemedicine location selected from the telemedicine location profiles, a requested medical specialty selected from the one or more medical specialties, a requested medical profile selected from the two or more medical profiles, a requested appointment length, a requested medical provider gender, an appointment urgency, a request for one or more medical peripherals, or a combination thereof.

3. The method of claim 1, further comprising assigning a role designation to a user, wherein the role designation comprises an institutional administrator, a department administrator, a technical administrator, a presenter, a healthcare provider, or a referring healthcare provider.

4. The method of claim 3, further comprising providing access to the computer system based on a hierarchical structure of the role assignment, an access and a function of the user.

5. The method of claim 1, wherein:
the referring healthcare provider device and the referred to healthcare provider device comprise one or more of a computer, a laptop, a handheld device, or a mobile device; and
the referring healthcare provider and the referred to healthcare provider each comprise one or more of a physician, nurse practitioner, physician assistance, nurse, nurse's aid, other healthcare professional, a healthcare coordinator or a healthcare staff.

6. The method of claim 1, wherein:
the referring healthcare provider device comprises more than one device; or
the referred to healthcare provider device comprises more than one device.

7. The method of claim 1, wherein the set of telemedicine encounter tasks comprise specifying a payment method, sending a request for a pre-appointment patient medical information, receiving the pre-appointment patient medical information, confirming the payment method, initiating the telemedicine encounter, completing the telemedicine encounter, sending the patient results, or receiving the patient results.

8. The method of claim 1, further comprising one or more of the following:
receiving a payment method from the referring healthcare provider device;
receiving a confirmation of the payment method from the referred to healthcare provider;
sending a request for a pre-appointment patient medical information to the referring healthcare provider device;
receiving the pre-appointment patient medical information from the referring healthcare provider device;
initiating the telemedicine encounter using the one or more videoconference links;
receiving a completion of the telemedicine encounter from the referred to healthcare provider device;
receiving the patient results from the referred to healthcare provider device; or
providing the patient results to the referring healthcare provider device.

9. The method of claim 1, further comprising automatically ranking the one or more appointment times based on one or more criteria comprising a capacity at the two or more telemedicine location profiles, the two or more healthcare provider profiles, or a utilization across a set of the resource pool profiles.

10. The method of claim 1, further comprising sharing one or more documents or files between the referring healthcare provider device and the referred to healthcare provider device via the computer system.

11. The method of claim 10, further comprising using the one or more documents or files for clinical support, administrative support, education, tutoring, training, credentialing of one or more of the resource pool profiles, or store and forward telemedicine consultations.

12. The method of claim 10, further comprising storing, distributing and processing the one or more documents for files in the telemedicine encounter or an evaluation of the telemedicine encounter.

13. The method of claim 1, further comprising:
receiving one or more documents or files from the referring healthcare provider device or the referred to healthcare provider device;
making the received one or more documents or files available for downloading to the referring healthcare provider device or the referred to healthcare provider device; and
removing the received one or more documents or files from the computer system after a preset amount of time or until the received one or more documents or files are downloaded.

14. The method of claim 1, wherein the resource pool profiles comprise healthcare facility profiles, healthcare department profiles, healthcare unit profiles, or healthcare organization profiles.

15. The method of claim 1, wherein the telemedicine location profiles comprise patient stations with video conference capabilities, and patient station peripherals for specific patient healthcare evaluations.

16. The method of claim 1, further comprising providing an assignment of the resource pool profiles in a list or calendar format.

17. The method of claim 1, wherein the computerized system is integrated into or communicably linked to an electronic medical record (EMR) system.

18. A system for scheduling and tracking a telemedicine encounter between a referring healthcare provider and a referred to healthcare provider, comprising:
an input/output interface;
a memory;
one or more processors communicably coupled to the input/output interface and the memory;
a database of resource pool profiles stored in the memory comprising two or more healthcare provider profiles, two or more telemedicine location profiles, and one or more patient profiles, wherein each healthcare provider profile is linked to one or more of the telemedicine location profiles and one or more medical specialties; and
the one or more processors:
receive a telemedicine scheduling request for a patient from a referring healthcare provider device via the input/output interface, the scheduling request comprising a patient data and one or more appointment parameters,
search the database of resource pool profiles for any medical provider profiles that match the one or more appointment parameters,
provide one or more appointment times that match the one or more appointment parameters to the referring healthcare provider device via the input/output interface, wherein each appointment time is linked to one or more of the healthcare provider profiles,
receive a selected appointment time from the one or more appointment times from the referring healthcare provider device via the input/output interface,
automatically send a booking alert to the referring healthcare provider device and a referred to healthcare provider device associated with the selected appointment time via the input/output interface, schedule the telemedicine encounter for the healthcare provider profile and the telemedicine location profile at the selected appointment time, and create an appointment timeline for the patient comprising a set of appointment tasks,
automatically create and provide one or more videoconference links for the telemedicine encounter to the referring healthcare provider device and the referred to healthcare provider device via the input/output interface, and
automatically track and send alerts upon completion of each of the set of telemedicine encounter tasks.

19. The system of claim 18, wherein the one or more processors perform one or more of the following:
receive a payment method from the referring healthcare provider device;
receive a confirmation of the payment method from the referred to healthcare provider;
send a request for a pre-appointment patient medical information to the referring healthcare provider device;
receive the pre-appointment patient medical information from the referring healthcare provider device;
initiate the telemedicine encounter using the one or more videoconference links;
receive a completion of the telemedicine encounter from the referred to healthcare provider device;
receive the patient results from the referred to healthcare provider device; or
provide the patient results to the referring healthcare provider device.

20. The system of claim 18, wherein the one or more processors:
receive one or more documents or files from the referring healthcare provider device or the referred to healthcare provider device;
make the received one or more documents or files available for downloading to the referring healthcare provider device or the referred to healthcare provider device; and
remove the received one or more documents or files from the computer system after a preset amount of time or until the received one or more documents or files are downloaded.
